(19) United States
(12) Patent Application Publication
(10) Pub. No.: US 2025/0271384 A1
(43) Pub. Date: Aug. 28, 2025

(54) MODULATION OF SENSOR FILM COMPOSITION FOR ENHANCED GAS DETECTION PERFORMANCE

(71) Applicant: HONDA MOTOR CO., LTD., Tokyo (JP)

(72) Inventors: Shutang CHEN, Raymond, OH (US); Gugang CHEN, Raymond, OH (US)

(21) Appl. No.: 18/590,563

(22) Filed: Feb. 28, 2024

(51) Int. Cl. G01N 27/12; G01N 33/00

(52) U.S. Cl. CPC G01N 27/127; G01N 33/0047; G01N 33/0054

(57) ABSTRACT

A chemoresistive film having a plurality of metal nanostructures, the plurality of metal nanostructures having a selective affinity to at least one volatile gas species, wherein the chemoresistive film exhibits a change in electrical resistance when the at least one volatile gas species is present in an environment of the chemoresistive film at a first concentration. Also include are sensors having the chemoresistive film.

### TECHNICAL FIELD

The present disclosure is directed to chemoresistive films for use in detection sensors.

### BACKGROUND

Chemoresistive techniques for monitoring gas concentrations have attracted recent focus due to their cost-effectiveness, operational simplicity, and seamless incorporation into compact devices, all of which are attributes that are optimal for gas surveillance systems. However, there remains a need in the art for chemoresistive sensor materials that provide acceptable sensitivity, selectivity, and overall efficacy. For example, chemoresistive sensors currently known in the art frequently manifest limitations in selectivity, often producing ambiguous responses to volatile gas species, thus complicating the precise identification and quantification of specific gaseous entities.

### SUMMARY

Disclosed herein is a chemoresistive film having a plurality of nanostructures, the plurality of nanostructures having a selective affinity to one or more selected chemical species. Also disclosed herein is a sensor having the chemoresistive film as described herein and methods of making the plurality of nanostructures, the chemoresistive film, and the sensor.

### BRIEF DESCRIPTION OF THE DRAWINGS

FIG. 1 shows an example sensor according to aspects of the present disclosure.

FIG. 2A shows an SEM image of the platinum branched nanoparticles prepared according to Example II(A).

FIG. 2B shows an SEM image of the platinum-nickel branched nanoparticles prepared according to Example II(B).

FIG. 2C shows an SEM image of the platinum-silver nanoparticles prepared according to Example II(C).

FIG. 2D shows an SEM image of the platinum-palladium branched nanoparticles prepared according to Example II(D)

FIG. 2E shows an SEM image of the platinum-gold branched nanoparticles prepared according to Example II(E).

FIG. 2F shows an SEM image of the platinum-copper branched nanoparticles prepared according to Example II(F).

FIG. 3A shows a TEM image of the platinum branched nanoparticles prepared according to Example II(A).

FIG. 3B shows a TEM image of the platinum-nickel branched nanoparticles prepared according to Example II(B).

FIG. 3C shows a TEM image of the platinum-silver nanoparticles prepared according to Example II(C).

FIG. 3D shows a TEM image of the platinum-palladium branched nanoparticles prepared according to Example II(D).

FIG. 3E shows a TEM image of the platinum-gold branched nanoparticles prepared according to Example II(E).

FIG. 3F shows a TEM image of the platinum-copper branched nanoparticles prepared according to Example II(F).

FIG. 4A shows an XRD pattern of the platinum branched nanoparticles prepared according to Example II(A).

FIG. 4B shows an XRD pattern of the platinum-nickel branched nanoparticles prepared according to Example II(B).

FIG. 4C shows an XRD pattern of the platinum-silver nanoparticles prepared according to Example II(C).

FIG. 4D shows an XRD pattern of the platinum-palladium branched nanoparticles prepared according to Example II(D).

FIG. 4E shows an XRD pattern of the platinum-gold branched nanoparticles prepared according to Example II(E).

FIG. 4F shows an XRD pattern of the platinum-copper branched nanoparticles prepared according to Example II(F).

FIG. 5 shows digital and SEM images of the sensors as described in Example V.

FIG. 6 shows SEM images of sensor films with different thicknesses and consistencies.

FIG. 7 shows the sensors as described in Example VI.

FIG. 8 shows the response of the platinum-nickel nanoparticle-based gas sensors to acetone and ammonia as described in Example VI.

FIG. 9A shows the results of the stability investigation of the sensors as described in Example VI.

FIG. 9B shows the results of the stability investigation of the sensors as described in Example VI.

FIG. 9C shows the results of the stability investigation of the sensors as described in Example VI.

FIG. 9D shows the results of the stability investigation of the sensors as described in Example VI.

FIG. 10A shows the response of the platinum-nickel nanoparticle-based sensors as described in Example VI.

FIG. 10B shows the response of the sensors as described in Example VI.

FIG. 10C shows the response of the sensors as described in Example VI.

FIG. 11A shows the response of the sensors as described in Example VI.

FIG. 11B shows the response of the sensors as described in Example VI.

FIG. 12 shows the energy levels of gas-phase NO in comparison to a metal surface as described in Example VI.

### DETAILED DESCRIPTION

Disclosed herein is a chemoresistive film having a plurality of nanostructures, the plurality of nanostructures having a selective affinity to one or more selected chemical species. Also disclosed herein is a sensor having the chemoresistive film as described herein and methods of making the plurality of nanostructures, the chemoresistive film, and the sensor.

As used herein, the term “chemoresistive” refers to materials that exhibit a change in electrical resistance in response to changes in their environment. It should thus be understood that a chemoresistive film as described herein refers to a film that exhibits a change in electrical resistance in response to a change in its environment, that is, any gas, liquid, and/or solid that is in contact with the chemoresistive film. As used herein, the term “film” refers to a structure having a relatively smaller thickness as compared with a relatively larger surface area, for example, a structure having a thickness of between about 500 nm and 2 μm, optionally between about 200 nm and 20 μm. A film may form a coating on a support structure and/or may be self-supportive (i.e., does not require a support structure to retain its spatial configuration).

The chemoresistive film according to the present disclosure may include a plurality of nanostructures. As used herein, the term “nanostructure” refers to a structure having at least one dimension on the nanoscale (i.e., at least on dimension between about 0.1 and 100 nm). It should be understood that “nanostructures” include, but are not limited to, nanosheets, nanotubes, nanoparticles, nanospheres, nanowires, nanocubes, and combinations thereof. A nanosheet may include a sheet having a thickness on the nanoscale. A nanotube may include a tube having a diameter on the nanoscale. A nanoparticle may include a particle wherein each spatial dimension thereof is on the nanoscale. A nanowire may include an elongated structure having a diameter on the nanoscale. A nanoparticle may include a particle wherein each spatial dimension thereof is on the nanoscale. A nanosphere may include a sphere wherein each spatial dimension thereof is on the nanoscale. A nanocube may include a cube wherein each spatial dimension thereof is on the nanoscale.

According to some aspects, the nanostructure may be branched, such as a branched nanoparticle. According to some aspects, the nanostructure may include a polyhedral nanoparticle. According to some aspects, the nanostructure may be hollow.

According to some aspects, the chemoresistive film may include a plurality of metal nanostructures formed from one or more metals. In some example aspects, the chemoresistive film may include a plurality of metal nanostructures having a first portion of metal nanostructures formed from a first metal or a first combination of metals, and a second portion of metal nanostructures formed from a second metal or a second combination of metals, the first metal and/or first combination of metals being different from the second metal and/or second combination of metals. It should be understood that the chemoresistive film may include third, fourth, fifth, sixth, or more portions of the plurality of metal nanostructures, wherein each of the portions is formed from a different metal or combination of metals from the one or more other portions.

Example metals useful according to the present disclosure include, but are not limited to, one or more group 1 to group 12 metals, that is, any alkali metal, alkaline earth metal, transition metal, or combination thereof. Non-limiting examples of metals useful according to the present disclosure include Pt, Ni, Pd, Au, Ag, Cu, Co, Fe, Zn, Cd, and combinations thereof.

Additionally or alternatively, the chemoresistive film may include a plurality of non-metal nanostructures formed from one or more non-metals. In some example aspects, the chemoresistive film may include a plurality of non-metal nanostructures having a first portion of non-metal nanostructures formed from a first non-metal or a first combination of non-metals, and a second portion of non-metal nanostructures formed from a second non-metal or a second combination of non-metals, the first non-metal and/or first combination of non-metals being different from the second non-metal and/or second combination of non-metals. It should be understood that the chemoresistive film may include third, fourth, fifth, sixth, or more portions of the plurality of non-metal nanostructures, wherein each of the portions is formed from a different non-metal or combination of non-metals from the one or more other portions.

Example non-metals useful according to the present disclosure include, but are not limited to, C, N, O, P, S, Se, and combinations thereof.

Additionally or alternatively, the chemoresistive film may include a plurality of nanostructures formed from one or more non-metals and one or more metals as described herein, hereinafter referred to as combination nanostructures. In some example aspects, the chemoresistive film may include a plurality of combination nanostructures having a first portion of combination nanostructures formed from a first combination of metals and non-metals, and a second portion of combination nanostructures formed from a second combination of metals and non-metals, the first combination of metals and non-metals being different from the second combination of metals and non-metals. It should be understood that the chemoresistive film may include third, fourth, fifth, sixth, or more portions of the plurality of combination nanostructures, wherein each of the portions is formed from a different combination of metals and non-metals from the one or more other portions.

According to some aspects, each portion of the plurality of nanostructures may have a selective affinity to one or more selected chemical species. As used herein, the term “chemical species” refers to a chemical compound or portion thereof (e.g., a functional group). It should be understood that the affinity as described herein is an affinity for interaction between the nanostructure and a chemical species. According to some aspects, the interaction may include chemisorption (i.e., the formation of covalent bond(s) between a nanostructure and a chemical species) and/or physisorption (i.e., adsorption between a nanostructure and a chemical species created by inter-molecular attractive forces). It should be understood that as used herein, a “selective affinity” refers to an affinity to only one or more selected chemical species. The selective affinity of each portion of the plurality of nanostructures to one or more selected chemical species may depend on the composition of the nanostructures (i.e., the chemical structure of the metal(s), non-metal(s), and/or combination thereof forming the nanostructures) and/or the shape of the nanostructures.

According to some aspects, an interaction between a nanostructure at least partially forming a chemoresistive film and one or more chemical species at least partially forming the chemoresistive film's environment may result in a certain change in the chemoresistive film's electrical resistance (ΔR). According to some aspects, ΔR may be determined by comparing the chemoresistive film's electrical resistance with the chemoresistive film's electrical resistance in a reference atmosphere (R₀) such that ΔR=Rₜ−R₀, with R₀ being the chemoresistive film's electrical resistance in a reference atmosphere, Rₜ being the chemoresistive film's electrical resistance after a certain period of time (t) in an atmosphere other than the reference atmosphere, and ΔR being the change in the chemoresistive film's electrical resistance. According to some aspects, the reference atmosphere may be an inert atmosphere (i.e., an atmosphere having only one or more inert gases) and/or air (i.e., the natural atmosphere surrounding the earth). It should be understood that ΔR may be an increase in electrical resistance or a decrease in electrical resistance compared with the chemoresistive film's electrical resistance in a reference atmosphere.

According to some aspects, a chemoresistive film's sensitivity to a chemical species at a certain concentration in an atmosphere may be represented as a response value, wherein the response value is ΔR/R₀ normalized to the background response under the reference atmosphere. According to some aspects of the present disclosure, the chemoresistive film may demonstrate a response value to one or more chemical species at a certain concentration of between about 0.01 and 5, optionally between about 0.01 and 1, optionally between about 1 and 2, optionally between about 2 and 3, optionally between about 3 and 4, optionally between about 4 and 5, optionally about 0.01, optionally about 0.02, optionally about 0.03, optionally about 0.04, optionally about 0.05, optionally about 0.06, optionally about 0.07, optionally about 0.08, optionally about 0.09, optionally about 0.1, optionally about 0.2, optionally about 0.3, optionally about 0.4, optionally about 0.5, optionally about 0.6, optionally about 0.7, optionally about 0.8, optionally about 0.9, optionally about 1.1, optionally about 1.2, optionally about 1.3, optionally about 1.4, optionally about 1.5, optionally about 1.6, optionally about 1.7, optionally about 1.8, optionally about 1.9, optionally about 2.1, optionally about 2, optionally about 2.2, optionally about 2.3, optionally about 2.4, optionally about 2.5, optionally about 2.6, optionally about 2.7, optionally about 2.8, optionally about 2.9, optionally about 3, optionally about 3.1, optionally about 3.2, optionally about 3.3, optionally about 3.4, optionally about 3.5, optionally about 3.6, optionally about 3.7, optionally about 3.8, optionally about 3.9, optionally about 4, optionally about 4.1, optionally about 4.2, optionally about 4.3, optionally about 4.4, optionally about 4.5, optionally about 4.6, optionally about 4.7, optionally about 4.8, optionally about 4.9, and optionally about 5. In some non-limiting example, the chemoresistive film may demonstrate the response value to the one or more chemical species at a concentration that is between about 0.01 and 10,000 ppm, optionally between about 0.1 and 1,000 ppm, and optionally between about 1 and 100 ppm, including all values therebetween, such as 10 ppm, 20 ppm, 30 ppm, 40 ppm, 50 ppm, 60 ppm, 70 ppm, 80 ppm, and 90 ppm, and/or at a concentration that is between about 0.01 and 10,000 ppb, optionally between about 0.1 and 1,000 ppb, and optionally between about 1 and 100 ppb, including all values therebetween, such as 10 ppb, 20 ppb, 30 ppb, 40 ppb, 50 ppb, 60 ppb, 70 ppb, 80 ppb, and 90 ppb, and/or a concentration that is no more than about 0.1 ppm, optionally no more than about 10 ppm, optionally no more than about 100 ppm, optionally no more than about 1,000 ppm, and optionally no more than about 10,000 ppm, and/or a concentration that is no more than about 0.1 ppb, optionally no more than about 10 ppb, optionally no more than about 100 ppb, optionally no more than about 1,000 ppb, and optionally no more than about 10,000 ppb.

According to some aspects, a certain ΔR may indicate the presence of one or more certain chemical species in an environment. In some examples, a certain ΔR may indicate that the one or more certain chemical species is present in an environment at or above a certain threshold concentration. As used herein, the term “threshold concentration” refers to a concentration of one or more chemical species in an environment sufficient to provide a certain ΔR in a chemoresistive film. Examples of threshold concentrations include, but are not limited to, a concentration that is between about 0.01 and 10,000 ppm, optionally between about 0.1 and 1,000 ppm, and optionally between about 1 and 100 ppm, including all values therebetween, such as 10 ppm, 20 ppm, 30 ppm, 40 ppm, 50 ppm, 60 ppm, 70 ppm, 80 ppm, and 90 ppm. Other examples of threshold concentrations include, but are not limited to, a concentration that is between about 0.01 and 10,000 ppb, optionally between about 0.1 and 1,000 ppb, and optionally between about 1 and 100 ppb, including all values therebetween, such as 10 ppb, 20 ppb, 30 ppb, 40 ppb, 50 ppb, 60 ppb, 70 ppb, 80 ppb, and 90 ppb. Other examples of threshold concentrations include, but are not limited to, a concentration that is no more than about 0.1 ppm, optionally no more than about 10 ppm, optionally no more than about 100 ppm, optionally no more than about 1,000 ppm, and optionally no more than about 10,000 ppm. Other examples of threshold concentrations include, but are not limited to, a concentration that is no more than about 0.1 ppb, optionally no more than about 10 ppb, optionally no more than about 100 ppb, optionally no more than about 1,000 ppb, and optionally no more than about 10,000 ppb.

It should be understood that by selecting one or more nanostructures having a selective affinity to one or more certain chemical species, a chemoresistive film for detecting the presence of the one or more selected chemical species in an environment at or above a certain threshold concentration may be provided. It should also be understood that the threshold concentration may be modified by modifying the composition and/or concentration of the one or more nanostructures in the chemoresistive film. That is, the sensitivity of a chemoresistive film toward one or more selected chemical species may be selected by selecting a certain type of nanostructures and/or a certain concentration thereof.

According to some aspects, a chemoresistive film's ΔR may be correlated with the concentration of one or more certain chemical species in an environment. In this way, by determining the chemoresistive film's ΔR, the concentration of the one or more chemical species in an environment may be determined.

According to some aspects, the chemoresistive film may include a plurality of nanostructures that have a selective affinity to one, two, three, four, or more certain chemical species. In some examples, the chemoresistive film may include a plurality of nanostructures with a first portion having a selective affinity to one, two, three, four, or more certain chemical species and a second portion having a selective affinity to one, two, three, four, or more certain chemical species, wherein the first portion and the second portion have a selective affinity to a different chemical species or a different combination of chemical species. It should be understood that the plurality of nanostructures may further include third, fourth, fifth, or more portions each having a selective affinity to one, two, three, four, or more certain chemical species as described herein, wherein the third, fourth, fifth, or more portions have a selective affinity to a different chemical species or a different combination of chemical species from the other portions.

In some non-limiting examples, the chemoresistive film of the present disclosure may include a plurality of nanostructures having a selective affinity to one or more gases. According to some aspects, the one or more gases may include one or more volatile gas species. In some non-limiting examples, the one or more gases may include acetone, isoprene, methanol, nitric oxide, ammonia, ethanol, formaldehyde, carbon dioxide, carbon monoxide, hydrogen, methane, propane, or a combination thereof. According to some aspects, the chemoresistive film may include a plurality of nanostructures that interact with the one or more gases sufficient to provide a certain ΔR when the one or more gases are present in the chemoresistive film's environment at or above a certain threshold concentration as described herein. According to some aspects, the chemoresistive film's ΔR may be correlated with the concentration of one or more certain gases in an environment.

The present disclosure is also directed to a sensor having a chemoresistive film as described herein. According to some aspects, the sensor may include a support on which one or more different chemoresistive films are provided. Example support materials include, but are not limited to, silica, tricalcium phosphate, hydroxyapatite, talc, magnesium stearate, calcium carbonate, and combinations thereof. The one or more chemoresistive films may be independently arranged in a circuit with two electrodes such that the one or more chemoresistive films function as a resistor between the two electrodes in the circuit. In this way, the sensor may allow for measuring the resistance of the one or more chemoresistive films in an environment as described herein.

According to some aspects, the sensor provides a signal when a ΔR is measured in one or more of the chemoresistive films. In this way, the sensor may provide a signal corresponding to a certain chemical species and/or a certain concentration of the certain chemical species in the sensor's environment. The signal may be any signal sufficient to signify the ΔR.

According to some aspects, the sensor of the present disclosure may include two or more different chemoresistive films, thus forming an array wherein each of the two or more different chemoresistive film is sensitive to a unique chemical species and/or a unique concentration of a chemical species.

In some non-limiting examples, the array may include a plurality of chemoresistive films such that the array is sensitive to at least 2 different volatile gas species, optionally at least 3 different volatile gas species, optionally at least 4 different volatile gas species, optionally at least 5 different volatile gas species, optionally at least 6 different volatile gas species, optionally at least 7 different volatile gas species, optionally at least 8 different volatile gas species, optionally at least 9 different volatile gas species, optionally at least 10 different volatile gas species, optionally at least 11 different volatile gas species, and optionally at least 12 different volatile gas species. It should be understood that the array may be sensitive to each volatile gas species at a single concentration and/or over a range of concentrations as described herein.

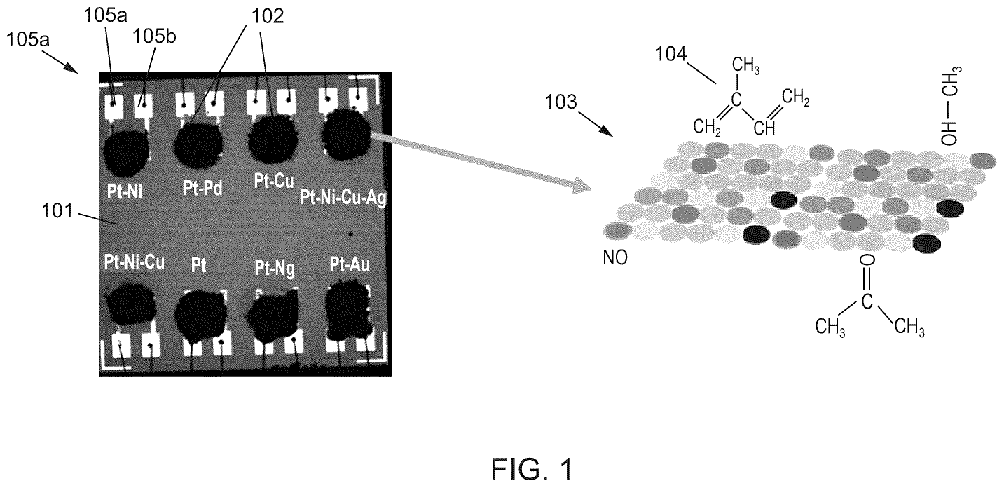

FIG. 1 shows an example sensor 100 according to aspects of the present disclosure. As shown in FIG. 1, sensor 100 includes a support 101 on which different chemoresistive films 102 are supported, each chemoresistive film 102 including a plurality of nanostructures formed from a different combination of metals. Each chemoresistive film 102 may be connected to a pair of leads, including a first lead 105a in communication with a first electrode and a second lead 105b in communication with a second electrode, sufficient to complete a circuit as described herein. FIG. 1 also shows a schematic view 103 of chemoresistive film 102 in an environment having a chemical species 104.

Also disclosed herein are methods of making the plurality of nanostructures, the chemoresistive film, and the sensors as described herein.

According to some aspects, the method may include providing a plurality of metal nanoparticles. In some non-limiting examples, at least a portion of the plurality of metal nanoparticles may include a single metal. Additionally or alternatively, at least a portion the plurality of metal nanoparticles may include two or more metals.

In one non-limiting example, the method may include providing a plurality of metal nanoparticles having a single metal. The method of this example may include providing a metal complex solution and heating the metal complex solution to an elevated temperature. The metal complex solution may include a complex of a metal and a nitrogen-containing compound. The metal may include any metal as described herein. Non-limiting examples of nitrogen-containing compounds include oleylamine (OLA), octadecylamine (ODA), hexadecylamine (HDA), dodecylamine (DDA), tetradecylamine (TDA), isomers thereof, derivatives thereof, or combinations thereof.

According to some aspects, the metal complex solution may be provided by combining a metal source with a nitrogen-containing compound. Example metal sources according to the present disclosure include, but are not limited to, metal chlorides, metal nitrates, metal acetylacetonate, metal salts, and combinations thereof.

According to some aspects, providing the metal complex solution may include mixing a metal source and a nitrogen containing compound at an elevated temperature. In some non-limiting examples, the elevated temperature may be between about 50 and 350° C., and optionally between about 100 and 300° C. According to some aspects, the metal source and the nitrogen containing compound may be mixed at the elevated temperature for a time period sufficient to provide a metal complex solution as described herein. In some non-limiting examples, the time period may be between 1 and 40 minutes, optionally between about 1 and 30 minutes, optionally between about 10 and 30 minutes, and optionally about 20 minutes.

According to some aspects, oxygen may be removed from the metal complex solution as known in the art. For example, the method may include combining the metal source and the nitrogen containing compound under an inert gas flow. Examples of inert gases include, but are not limited to, nitrogen gas, argon gas, and combinations thereof.

In another non-limiting example, the method may include providing a plurality of bimetallic nanoparticles. The method of this example may include combining a first metal complex solution with a second metal complex solution, wherein each of the first metal complex solution and the second metal complex solution independently includes a complex of a metal and a nitrogen-containing compound. Each of the metals may include any metal as described herein. Non-limiting examples of nitrogen-containing compounds include those described herein.

According to some aspects, each metal complex solution may be provided by combining a metal source with a nitrogen-containing compound. Example metal sources according to the present disclosure include, but are not limited to, metal chlorides, metal nitrates, metal acetylacetonate, metal salts, and combinations thereof.

For example, when the first metal complex solution includes platinum, the metal source may include chloroplatinic acid hexahydrate, sodium hexachloroplatinate hexahydrate, platinum chloride, platinum acetylacetonate, or a combination thereof. In this example, the first metal complex may include Pt-TDA, Pt-OLA, Pt-HDA, Pt-ODA, Pt-DDA, or a combination thereof.

In another example, when the second metal complex solution includes nickel, the metal source may include nickel acetylacetonate, nickel chloride, nickel nitrate, nickel oxide, or a combination thereof. In this example, the second metal complex may include Ni-TDA, Ni-OLA, Ni-HDA, Ni-ODA, Ni-DDA, or a combination thereof.

It should be understood, however, that the disclosure is not limited to platinum and nickel. Rather, the metal source may include any metal as described herein.

According to some aspects, providing the first and/or second metal complex solution may include mixing a metal source and a nitrogen containing compound at an elevated temperature. In some non-limiting examples, the elevated temperature may be between about 100 and 300° C., optionally between about 150 and 250° C., and optionally about 100° C. According to some aspects, the metal source and the nitrogen containing compound may be mixed at the elevated temperature for a time period sufficient to provide a metal complex solution as described herein. In some non-limiting examples, the time period may be between 1 and 30 minutes, optionally between about 1 and 20 minutes, optionally between about 1 and 15 minutes, optionally between about 1 and 10 minutes, optionally about 10 minutes, optionally about 9 minutes, optionally about 8 minutes, optionally about 7 minutes, optionally about 6 minutes, and optionally about 5 minutes.

According to some aspects, the atomic molar ratio of the first metal source to the first nitrogen containing compound combined to provide the first metal complex solution may be between about 1:1 and 1:1000, optionally between about 1:10 and 1:500, optionally between about 1:50, and 1:300, optionally between about 1:80 and 1:150.

According to some aspects, the atomic molar ratio of the second metal source to the second nitrogen containing compound combined to provide the second metal complex solution may be between about 1:1 and 1:500, optionally between about 1:10 and 1:300, optionally between about 1:50 and 1:200, optionally between about 1:80 and 1:150.

According to some aspects, oxygen may be removed from the first metal complex solution and/or the second metal complex solution as known in the art. For example, the method may include combining the first metal source and the first nitrogen containing compound under an inert gas flow. Additionally or alternatively, the method may include combining the second metal source and the second nitrogen containing compound under an inert gas flow. Examples of inert gases include those described herein.

The method may include combining the first metal complex solution with the second metal complex solution. According to some aspects, the first metal complex solution and the second metal complex solution may be combined within a solution having one or more other components, such as one or more metal sources and one or more nitrogen containing compounds as discloses herein. In one non-limiting example, the method may include combining a first metal source with a first nitrogen containing compound, removing oxygen, and heating to an elevated temperature to provide a reaction solution. According to some aspects, the elevated temperature may be between 10° and 300° C., optionally between about 150 and 250° C., and optionally about 200° C.

The method may further include, simultaneously or sequentially, adding the first metal complex solution to the reaction solution and adding the second metal complex solution to the reaction solution. The first metal complex solution and/or the second metal complex solution may independently be added to the reaction solution at a temperature between about 20 and 350° C., optionally between about 20 and 250° C., optionally between about 20 and 22° C., and optionally between about 150 and 250° C.

The method of the present disclosure may further include one or more washing steps to provide a plurality of metal or bimetallic nanoparticles as disclosed herein. According to some aspects, the one or more washing steps may include combining the reaction solution with a solvent, centrifuging the solution, and/or discarding the supernatant as known in the art. According to some aspects, the reaction solution may be cooled prior to one or more of the one or more washing steps. For example, the reaction solution may be cooled by at least about 20° C., optionally at least about 30° C., at least about 40° C., at least about 50° C., at least about 60° C., at least about 70° C., at least about 80° C., at least about 90° C., at least about 100° C., at least about 110° C., or at least about 120° C.

According to some aspects, the one or more washing steps may include combining the reaction solution with a solvent. In some non-limiting examples, the solvent may include at least one hydrophobic solvent, at least one organic solvent, or a combination thereof. Example hydrophobic solvents useful according to the present disclosure include, but are not limited to, hexane, benzene, toluene, xylene, chlorobenzene, dichlorobenzene, trichlorobenzene, cyclohexane, carbon tetrachloride chloroform, and combinations thereof. Example organic solvents useful according to the present disclosure include, but are not limited to, aromatic compounds (e.g., benzene, toluene), alcohols (e.g. ethanol, methanol), esters, ethers, ketones (e.g., acetone), amines, nitrated and halogenated hydrocarbons, and combinations thereof. It should be understood that the resulting product may be stored in a solvent as described herein, as known in the art.

According to some aspects, the method may include providing a chemoresistive film as described herein. In some non-limiting examples, the method may include providing a solution having a plurality of nanostructures and a solvent, applying the solution onto a support, drying the solvent to provide the chemoresistive film, and optionally removing the chemoresistive film from the support.

In some non-limiting examples, the method may include, prior to providing the solution, transitioning a plurality of metal nanostructures into a plurality of hydrophilic metal nanostructures such that the solution applied onto the support contains a plurality of hydrophilic metal nanostructures and a solvent. In this way, the method may provide a smooth, uniform chemoresistive film.

According to some aspects, transitioning a plurality of metal nanostructures into a plurality of hydrophilic metal nanostructures may include replacing one or more surface ligands of the plurality of metal nanostructures with a hydrophilic component. For example, as described herein, the method of the present disclosure may include heating a first metal complex solution and/or a second metal complex solution to an elevated temperature sufficient to provide plurality of metal nanostructures. In this example, the resulting plurality of metal nanostructures may include one or more surface ligands associated with the nitrogen-containing compound(s) of the first metal complex and/or the second metal complex. The method of the present disclosure may include replacing at least a portion of the surface ligands with a hydrophilic component.

According to some aspects, replacing at least a portion of the surface ligands with a hydrophilic component may include combining a plurality of metal nanostructures with a hydrophilic component at a certain ratio to provide a mixture, mixing the mixture for a certain mixing period, centrifuging the mixture at a certain centrifuge speed for a certain centrifuge period, and/or discarding the supernatant. In this way, at least a portion of the hydrophilic component may coordinate with metal atoms of the plurality of metal nanostructures sufficient to replace one or more surface ligands thereof.

According to some aspects, the ratio may be a molar ratio of metal atoms to hydrophilic component molecules. In some non-limiting examples, the molar ratio may be between about 1:1 to 1:100, optionally between about 1:5 and 1:80, optionally between about 1:10 and 1:60, and optionally between about 1:20 and 1:40. According to some aspects, the mixing period may be between about 1 minute and 24 hours, optionally between about 6 and 18 hours, and optionally about 12 hours. According to some aspects, the centrifuge speed may be between about 1000 and 7000 rpm, optionally between about 3000 and 5000 rpm, optionally about 1000 rpm, optionally about 2000 rpm, optionally about 3000 rpm, optionally about 4000 rpm, optionally about 5000 rpm, optionally about 6000 rpm, and optionally about 7000 rpm. According to some aspects, the centrifuge period may be between about 30 seconds and 10 minutes, optionally between about 30 seconds and 5 minutes, optionally between about 3 minutes and 5 minutes, and optionally about 2 minutes. In some non-limiting examples, the concentration of hydrophilic component molecules in coordination with metal atoms of the plurality of metal nanostructures may selected by selecting a certain centrifuge speed and/or centrifuge period. In some non-limiting examples, centrifuging and discarding the supernatant may be repeated once, twice, or more times using a solvent for each repeated centrifuging period. Example solvents include those described herein, such as water, ethanol, methanol, acetone, and combinations thereof.

Non-limiting examples of hydrophilic components according to the present disclosure include acids (e.g., acetic acid), sulfuric acid, phosphoric acid, nitric acid, and combinations thereof.

The present disclosure is also directed to methods of using the sensors as described herein. According to some aspects, the method may include providing a sensor as described herein, measuring the electrical resistance of the chemoresistive film comprised by the sensor in a reference atmosphere, contacting the sensor with an environment to be analyzed, and measuring a change in the chemoresistive film's electrical resistance (ΔR).

While the aspects described herein have been described in conjunction with the example aspects outlined above, various alternatives, modifications, variations, improvements, and/or substantial equivalents, whether known or that are or may be presently unforeseen, may become apparent to those having at least ordinary skill in the art. Accordingly, the example aspects, as set forth above, are intended to be illustrative, not limiting. Various changes may be made without departing from the spirit and scope of the disclosure. Therefore, the disclosure is intended to embrace all known or later-developed alternatives, modifications, variations, improvements, and/or substantial equivalents.

Thus, the claims are not intended to be limited to the aspects shown herein, but are to be accorded the full scope consistent with the language of the claims, wherein reference to an element in the singular is not intended to mean “one and only one” unless specifically so stated, but rather “one or more.” All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed as a means plus function unless the element is expressly recited using the phrase “means for.”

Herein, the recitation of numerical ranges by endpoints (e.g. between about 50:1 and 1:1, between about 100 and 500° C., between about 1 minute and 60 minutes) include all numbers subsumed within that range, for example, between about 1 minute and 60 minutes includes 21, 22, 23, and 24 minutes as endpoints within the specified range. Thus, for example, ranges 22-36, 25-32, 23-29, etc. are also ranges with endpoints subsumed within the range 1-60 depending on the starting materials used, temperature, specific applications, specific embodiments, or limitations of the claims if needed. The Examples and methods disclosed herein demonstrate the recited ranges subsume every point within the ranges because different synthetic products result from changing one or more reaction parameters. Further, the methods and Examples disclosed herein describe various aspects of the disclosed ranges and the effects if the ranges are changed individually or in combination with other recited ranges.

Further, the word “example” is used herein to mean “serving as an example, instance, or illustration.” Any aspect described herein as “example” is not necessarily to be construed as preferred or advantageous over other aspects. Unless specifically stated otherwise, the term “some” refers to one or more. Combinations such as “at least one of A, B, or C,” “at least one of A, B, and C,” and “A, B, C, or any combination thereof” include any combination of A, B, and/or C, and may include multiples of A, multiples of B, or multiples of C. Specifically, combinations such as “at least one of A, B, or C,” “at least one of A, B, and C,” and “A, B, C, or any combination thereof” may be A only, B only, C only, A and B, A and C, B and C, or A and B and C, where any such combinations may contain one or more member or members of A, B, or C. Nothing disclosed herein is intended to be dedicated to the public regardless of whether such disclosure is explicitly recited in the claims.

As used herein, the term “about” and “approximately” are defined to being close to as understood by one of ordinary skill in the art. In one non-limiting embodiment, the term “about” and “approximately” are defined to be within 10%, preferably within 5%, more preferably within 1%, and most preferably within 0.5%.

The examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, dimensions, etc.) but some experimental errors and deviations should be accounted for.

### EXAMPLES

### Example I(A): Synthesis of Pt-OLA Precursor Stock Solution

51.7 mg of chloroplatinic acid hexahydrate (0.1 mmol) and 2.0 mL oleylamine (OLA, 70%) were heated and shaken for 5 minutes. It was determined that acceptable amounts of chloroplatinic acid hexahydrate may vary from 5.17 mg to 2.068 g, and acceptable amounts of OLA also increase in corresponding fashion.

### Example I(B): Synthesis of Ni-OLA Precursor Stock Solution

0.02 mmol of nickel acetylacetonate and 2.0 mL of OLA were heated and shaken for 5 minutes. It was determined that acceptable amounts of nickel acetylacetonate may vary from 0.005 mmol to 1 mmol, and acceptable amounts of OLA also increase in corresponding fashion.

### Example I(C): Synthesis of Ag-OLA Precursor Stock Solution

0.02 mmol of silver nitrate and 2.0 mL of OLA were heated and shaken for 5 minutes.

### Example I(D): Synthesis of Pd-OLA Precursor Stock Solution

0.02 mmol of palladium acetylacetonate and 2.0 mL of OLA were heated and shaken for 5 minutes.

### Example I(E): Synthesis of Au-OLA Precursor Stock Solution

0.02 mmol of tetrachloroauric(III) acid trihydrate and 2.0 mL of OLA were heated and shaken for 5 minutes.

### Example I(F): Synthesis of Cu-OLA Precursor Stock Solution

0.02 mmol of copper acetylacetonate and 2.0 mL of OLA were heated and shaken for 5 minutes.

### Example II(A): Synthesis of Platinum Branched Nanoparticles

First, 5.0 g of hexadecylamine and 51.7 mg of chloroplatinic acid hexahydrate were loaded in a 50 mL three-neck flask where oxygen was removed through Ar blowing for 20 minutes. The reaction solution was heated to 200° C. and turned light gray, then kept for 20 minutes at 200° C., The reaction solution was then cooled to 100° C., and 5 mL of hexane (99%) and 5 mL of ethanol (200 proof) were injected. The products were separated by centrifuging at 2000 rpm for one minute. The supernatant was discarded. 10 mL of hexane was then added to the sediment, and the mixture was centrifuged at 4000 rpm for 5 minutes. The washing procedure was repeated twice to remove unreacted precursors and surfactant. The resulting platinum branched nanoparticles were stored in hydrophobic solvents (e.g., hexane, toluene, and chloroform) before characterization.

### Example II(B): Synthesis of Platinum-Nickel Branched Nanoparticles

First, 5.0 g of hexadecylamine and 51.7 mg of chloroplatinic acid hexahydrate were loaded in a 50 mL three-neck flask where oxygen was removed through Ar blowing for 20 minutes. When the reaction solution was heated to 200° C. and turned light gray, 3.0 mL of the Pt-OLA precursor solution from Example I(A) was injected into the flask under Ar flow. After 20 minutes at 200° C., the Ni-OLA precursor solution from Example I(B) was slowly injected (1.0 mL/min) and retained for another 20 minutes at 200° C. The reaction solution was then cooled to 80° C., and 5 mL of hexane (99%) and 5 mL of ethanol (200 proof) were injected. The products were separated by centrifuging at 2000 rpm for one minute. The supernatant was discarded. 10 mL of hexane was then added to the sediment, and the mixture was centrifuged at 4000 rpm for 5 minutes. The washing procedure was repeated twice to remove unreacted precursors and surfactant. The resulting platinum-nickel branched nanoparticles were stored in hydrophobic solvents (e.g., hexane, toluene, and chloroform) before characterization.

### Example II(C): Synthesis of Platinum-Silver Branched Nanoparticles

First, 5.0 g of hexadecylamine and 51.7 mg of chloroplatinic acid hexahydrate were loaded in a 50 mL three-neck flask where oxygen was removed through Ar blowing for 20 minutes. When the reaction solution was heated to 200° C. and turned light gray, 3.0 mL of the Pt-OLA precursor solution from Example I(A) was injected into the flask under Ar flow. After 20 minutes at 200° C., the Ag-OLA precursor solution from Example I(C) was slowly injected (1.0 mL/min) and retained for another 20 minutes at 200° C. The reaction solution was then cooled to 80° C., and 5 mL of hexane (99%) and 5 mL of ethanol (200 proof) were injected. The products were separated by centrifuging at 2000 rpm for one minute. The supernatant was discarded. 10 mL of hexane was then added to the sediment, and the mixture was centrifuged at 4000 rpm for 5 minutes. The washing procedure was repeated twice to remove unreacted precursors and surfactant. The resulting platinum-silver branched nanoparticles were stored in hydrophobic solvents (e.g., hexane, toluene, and chloroform) before characterization.

### Example II(D): Synthesis of Platinum-Palladium Branched Nanoparticles

First, 5.0 g of hexadecylamine and 51.7 mg of chloroplatinic acid hexahydrate were loaded in a 50 mL three-neck flask where oxygen was removed through Ar blowing for 20 minutes. When the reaction solution was heated to 200° C. and turned light gray, 3.0 mL of the Pt-OLA precursor solution from Example I(A) was injected into the flask under Ar flow. After 20 minutes at 200° C., the Pd-OLA precursor solution from Example I(D) was slowly injected (1.0 mL/min) and retained for another 20 minutes at 200° C. The reaction solution was then cooled to 80° C., and 5 mL of hexane (99%) and 5 mL of ethanol (200 proof) were injected. The products were separated by centrifuging at 2000 rpm for one minute. The supernatant was discarded. 10 mL of hexane was then added to the sediment, and the mixture was centrifuged at 4000 rpm for 5 minutes. The washing procedure was repeated twice to remove unreacted precursors and surfactant. The resulting platinum-palladium branched nanoparticles were stored in hydrophobic solvents (e.g., hexane, toluene, and chloroform) before characterization.

### Example II(E): Synthesis of Platinum-Gold Branched Nanoparticles

First, 5.0 g of hexadecylamine and 51.7 mg of chloroplatinic acid hexahydrate were loaded in a 50 mL three-neck flask where oxygen was removed through Ar blowing for 20 minutes. When the reaction solution was heated to 200° C. and turned light gray, 3.0 mL of the Pt-OLA precursor solution from Example I(A) was injected into the flask under Ar flow. After 20 minutes at 200° C., the Au-OLA precursor solution from Example I(E) was slowly injected (1.0 mL/min) and retained for another 20 minutes at 200° C. The reaction solution was then cooled to 80° C., and 5 mL of hexane (99%) and 5 mL of ethanol (200 proof) were injected. The products were separated by centrifuging at 2000 rpm for one minute. The supernatant was discarded. 10 mL of hexane was then added to the sediment, and the mixture was centrifuged at 4000 rpm for 5 minutes. The washing procedure was repeated twice to remove unreacted precursors and surfactant. The resulting platinum-gold branched nanoparticles were stored in hydrophobic solvents (e.g., hexane, toluene, and chloroform) before characterization.

### Example II(F): Synthesis of Platinum-Copper Branched Nanoparticles

First, 5.0 g of hexadecylamine and 51.7 mg of chloroplatinic acid hexahydrate were loaded in a 50 mL three-neck flask where oxygen was removed through Ar blowing for 20 minutes. When the reaction solution was heated to 200° C. and turned light gray, 3.0 mL of the Pt-OLA precursor solution from Example I(A) was injected into the flask under Ar flow. After 20 minutes at 200° C., the Cu-OLA precursor solution from Example I(F) was slowly injected (1.0 mL/min) and retained for another 20 minutes at 200° C. The reaction solution was then cooled to 80° C., and 5 mL of hexane (99%) and 5 mL of ethanol (200 proof) were injected. The products were separated by centrifuging at 2000 rpm for one minute. The supernatant was discarded. 10 mL of hexane was then added to the sediment, and the mixture was centrifuged at 4000 rpm for 5 minutes. The washing procedure was repeated twice to remove unreacted precursors and surfactant. The resulting platinum-copper branched nanoparticles were stored in hydrophobic solvents (e.g., hexane, toluene, and chloroform) before characterization.

### Example IV: Characterization Branched Nanoparticles

The surface morphologies of the nanoparticles prepared according to Examples II(A)-II(F) were investigated by a scanning electron microscope (SEM, QUANTA FEG 650) from FEI with a field emitter as electron source. A Bruker D8 Advance X-ray diffractometer with Cu Kα radiation operated at a tube voltage of 40 kV and a current of 40 mA was used to obtain X-ray diffraction (XRD) patterns. Transmission electron microscopy (TEM) images were captured using an FEI Tecnai 20 microscope with an accelerating voltage of 200 kV.

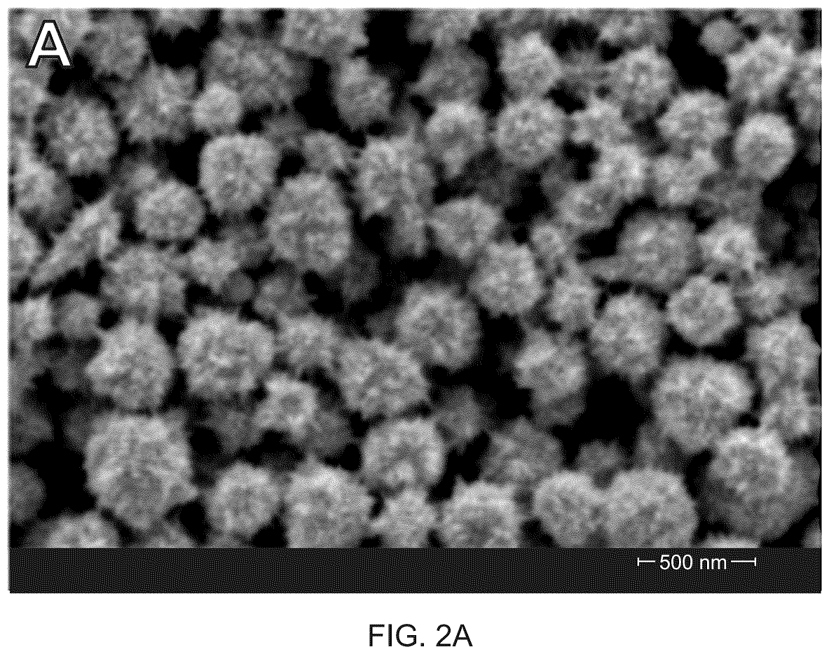

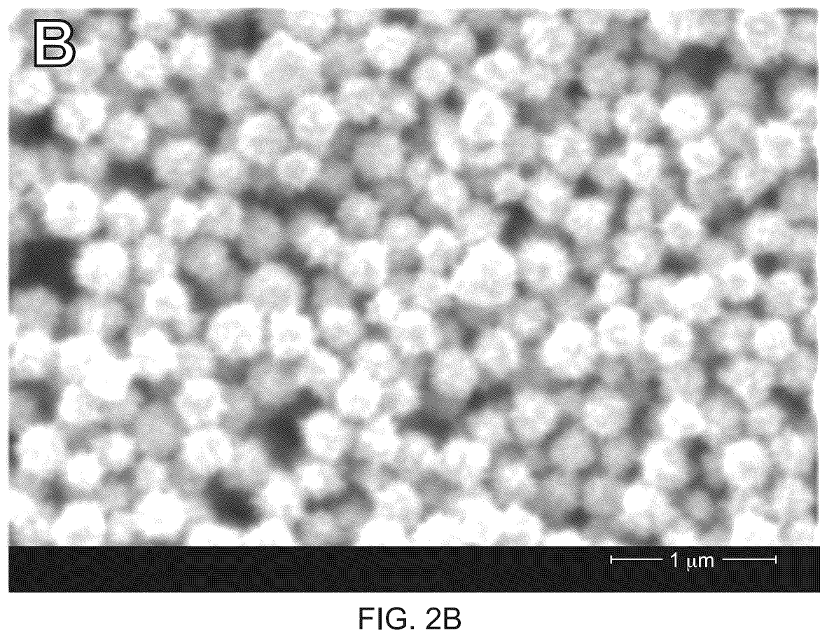

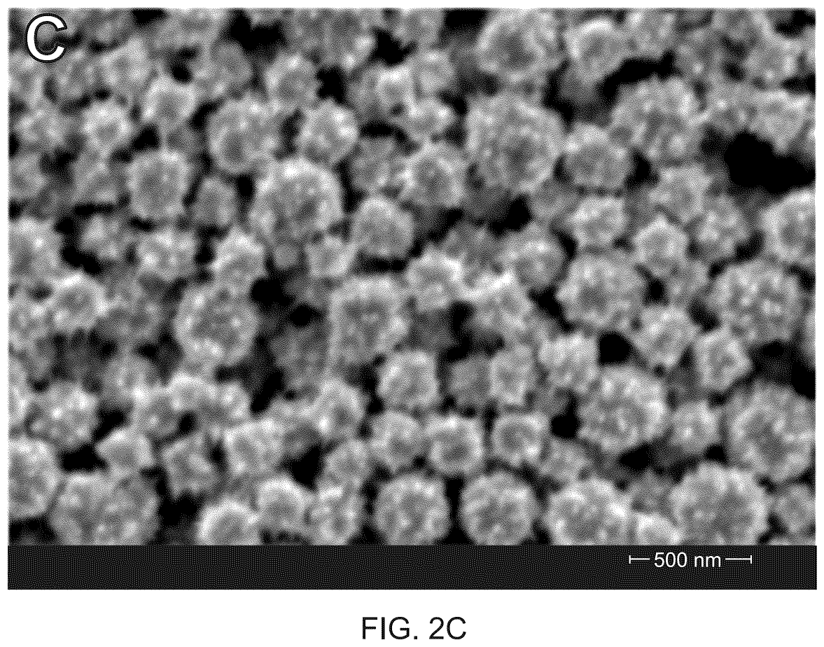

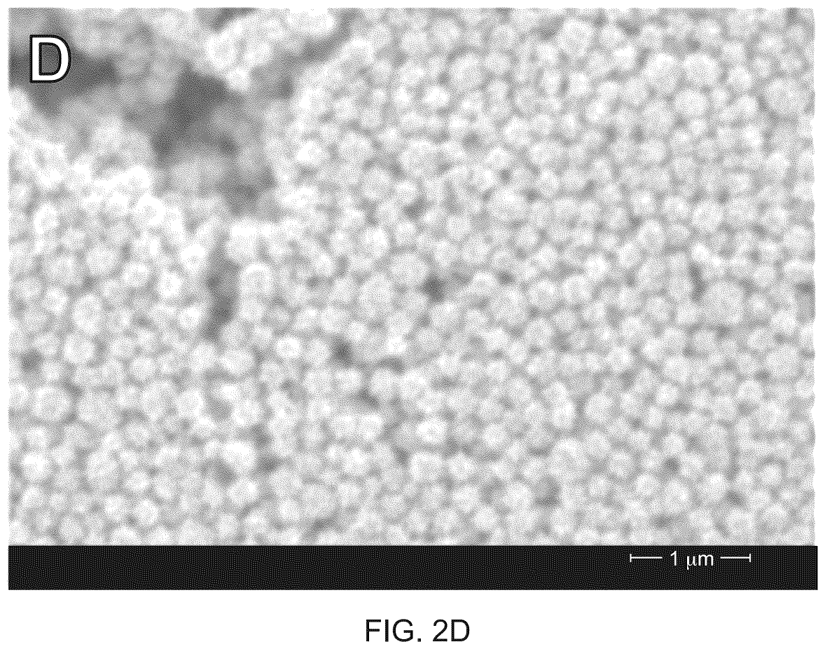

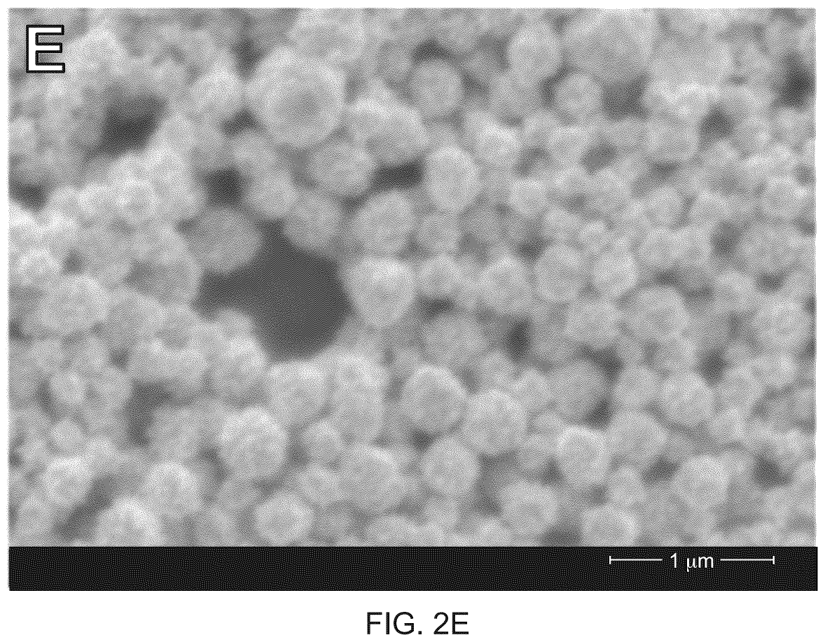

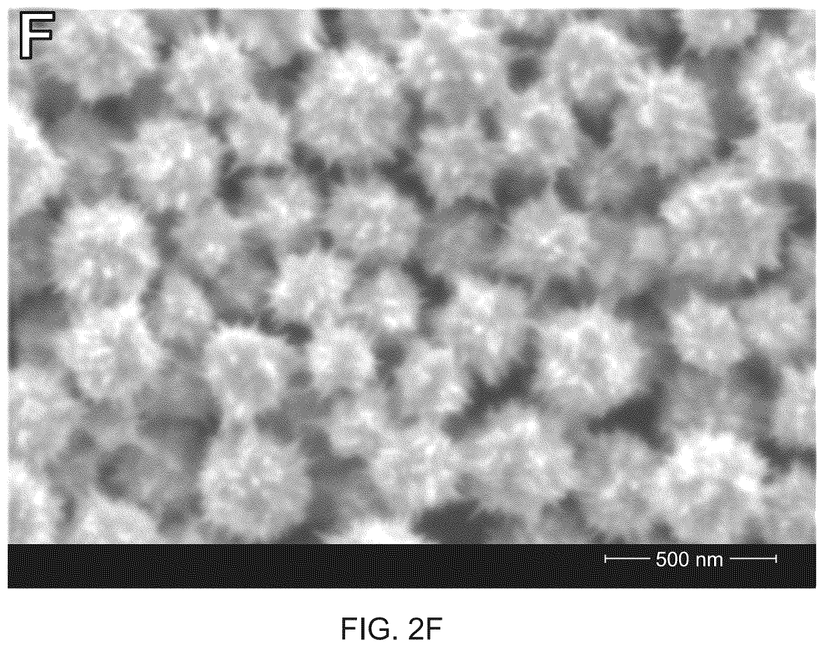

FIGS. 2A-F show that the nanoparticles prepared according to Examples II(A)-II(F) had branch-shaped surfaces with a diameter of about a few hundred nanometers, which provide a large number of adsorption sites for the metal particles. In particular, FIG. 2A shows an SEM image of the platinum branched nanoparticles prepared according to Example II(A), FIG. 2B shows an SEM image of the platinum-nickel branched nanoparticles prepared according to Example II(B), FIG. 2C shows an SEM image of the platinum-silver nanoparticles prepared according to Example II(C), FIG. 2D shows an SEM image of the platinum-palladium branched nanoparticles prepared according to Example II(D), FIG. 2E shows an SEM image of the platinum-gold branched nanoparticles prepared according to Example II(E), and FIG. 2F shows an SEM image of the platinum-copper branched nanoparticles prepared according to Example II(F).

To affirm the branched structure of the metal nanoparticles in high resolution, hydrophilic metal nanostructure inks were prepared by dispersing 10 mg of the nanoparticles prepared according to Examples II(A)-II(F) in 2.0 mL of acetic acid each. The mixtures were stirred for 12 hours. The products were then separated by centrifuging at 4000 rpm for 2 minutes, and the supernatant was discarded. Then, 5 mL of DI water was added to the sediment, and the mixture was again centrifuged at 4000 rpm for 5 minutes. This washing procedure was repeated twice to remove residue acid and surfactant. The resulting hydrophilic metal nanostructure inks were then delivered to carbon-coated copper grid surfaces and dried at room temperature to provide films.

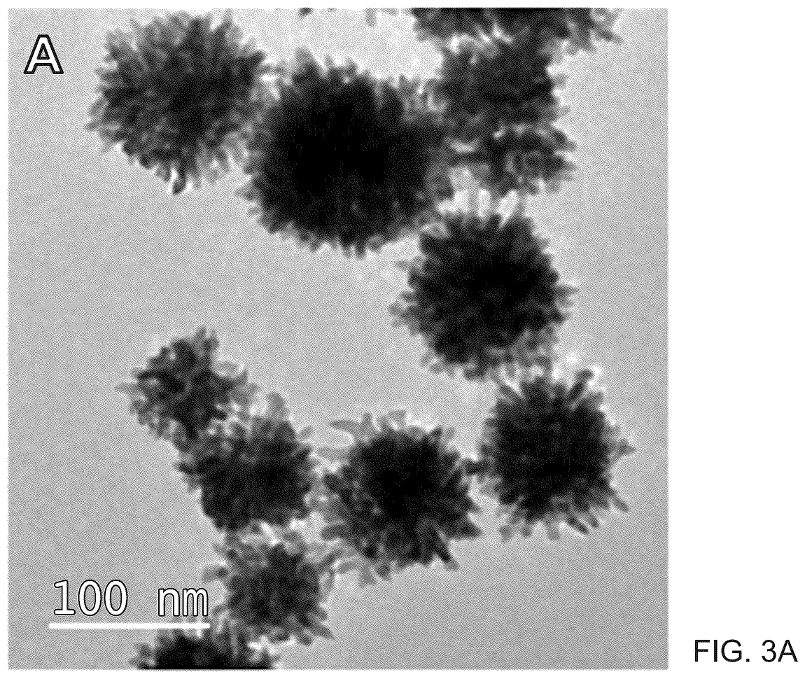

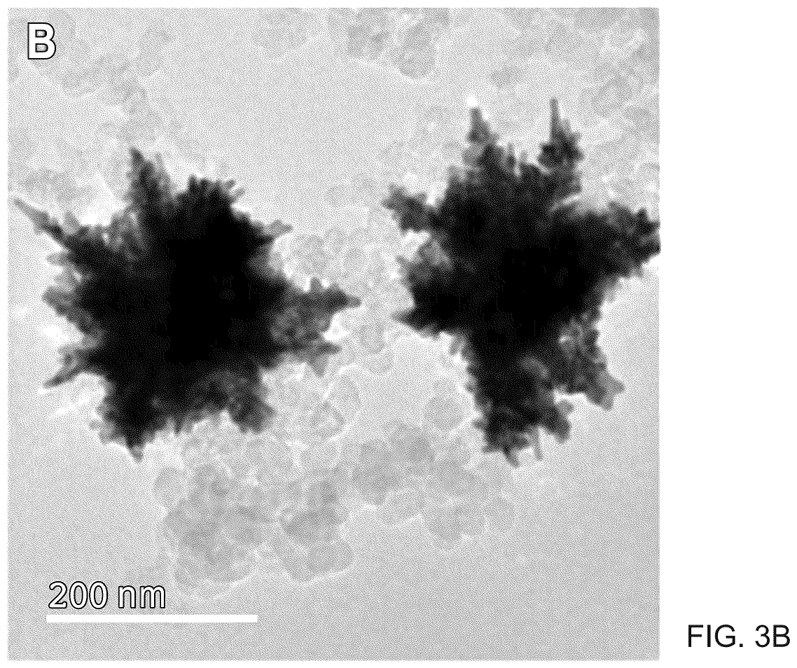

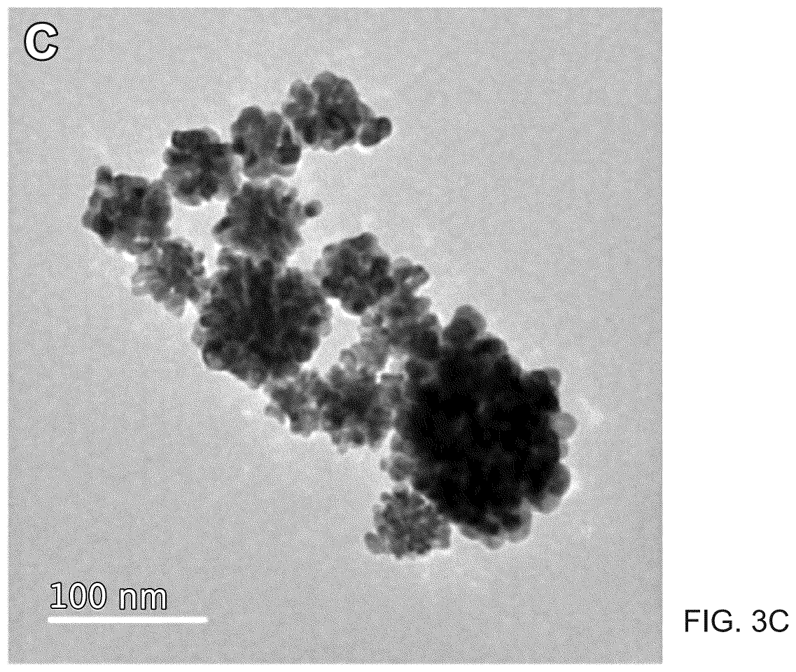

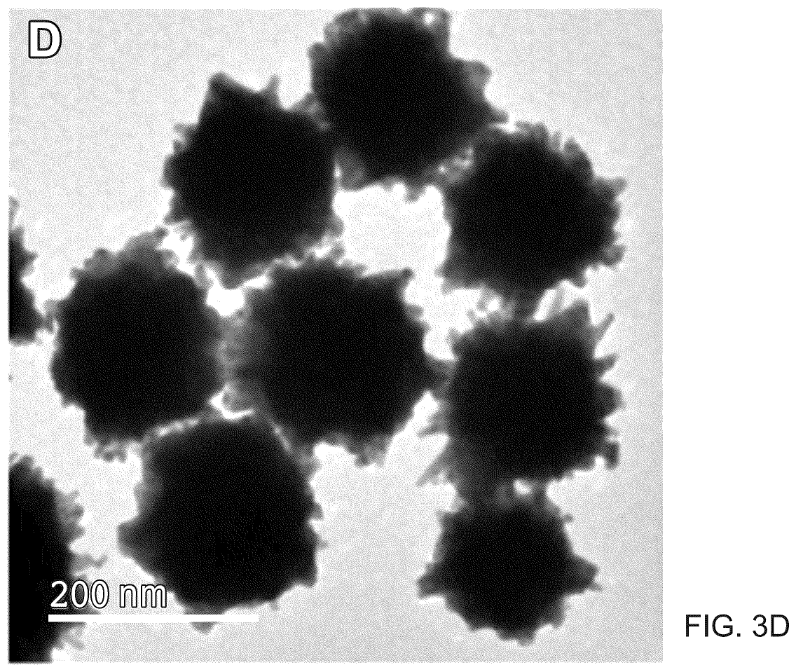

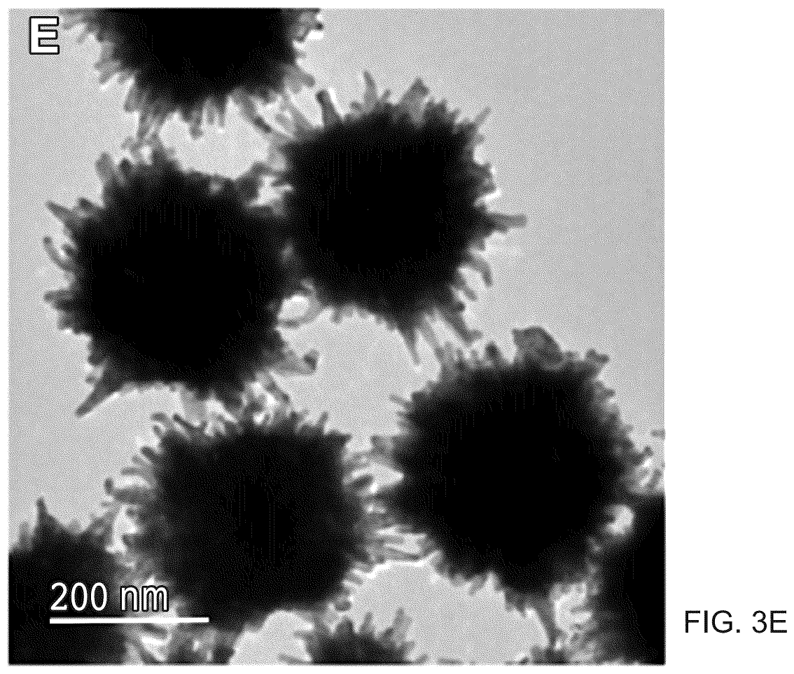

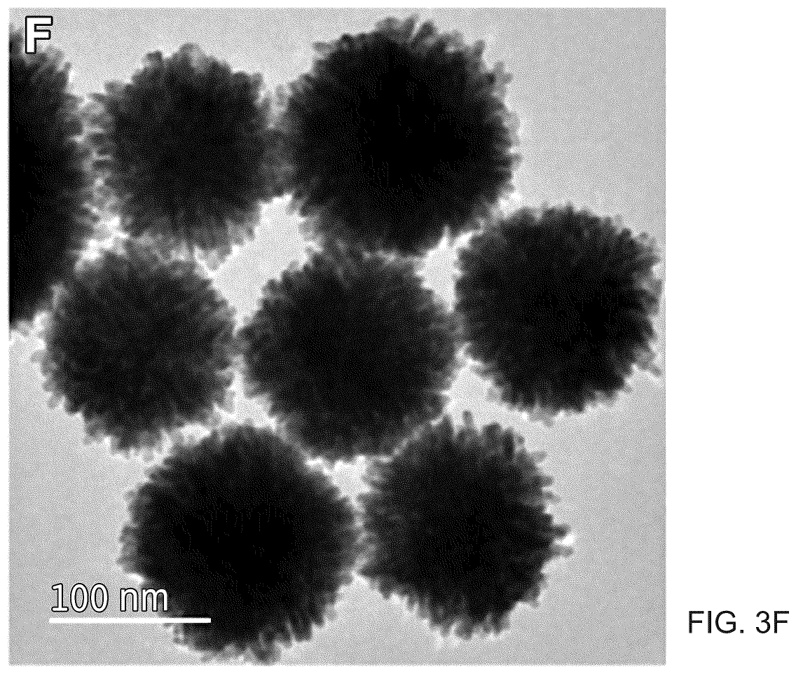

The transmission electron microscopy (TEM) images of the resulting films were captured using an FEI Tecnai 20 microscope with an accelerating voltage of 200 kV, as shown in FIGS. 3A-F. In particular, FIG. 3A shows a TEM image of the platinum branched nanoparticles prepared according to Example II(A), FIG. 3B shows a TEM image of the platinum-nickel branched nanoparticles prepared according to Example II(B), FIG. 3C shows a TEM image of the platinum-silver nanoparticles prepared according to Example II(C), FIG. 3D shows a TEM image of the platinum-palladium branched nanoparticles prepared according to Example II(D), FIG. 3E shows a TEM image of the platinum-gold branched nanoparticles prepared according to Example II(E), and FIG. 3F shows a TEM image of the platinum-copper branched nanoparticles prepared according to Example II(F). FIGS. 3A-F confirm the presence of branches measuring 10-20 nm each. This structural feature is significant as it provides favorable physisorption sites for the adsorption of VOCs (i.e., volatile organic compounds).

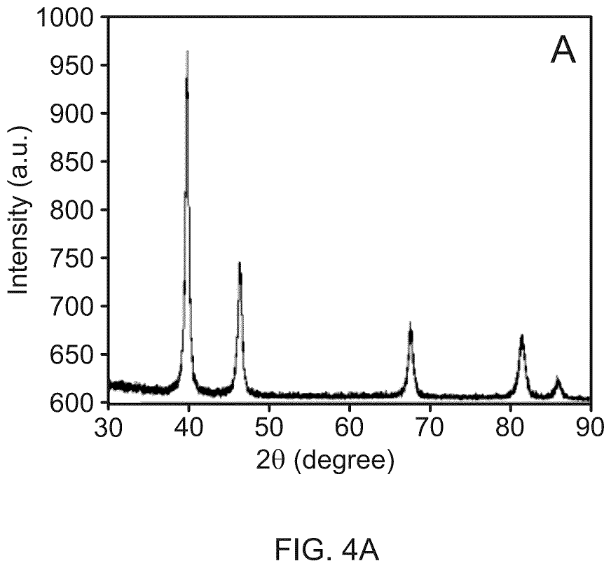

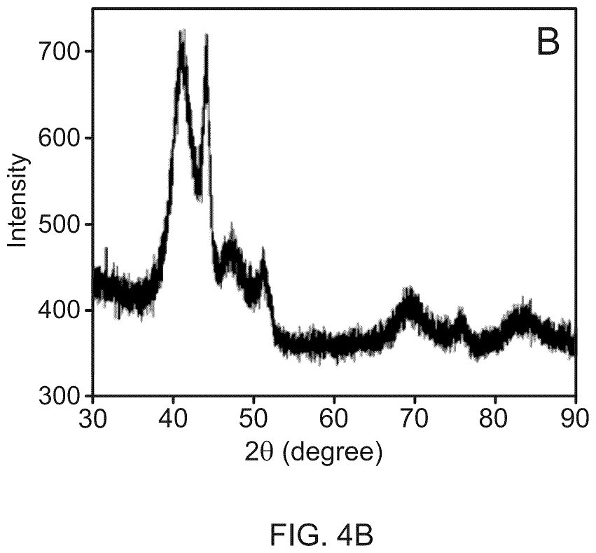

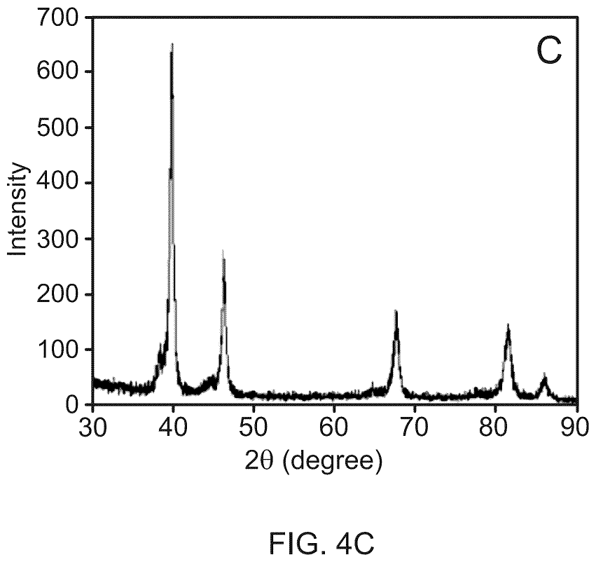

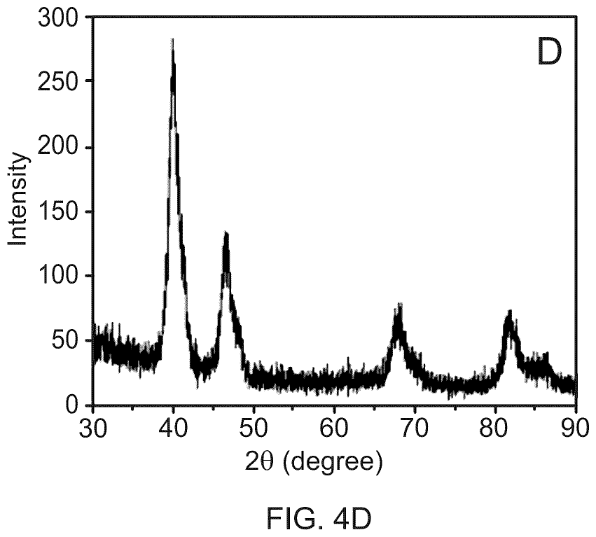

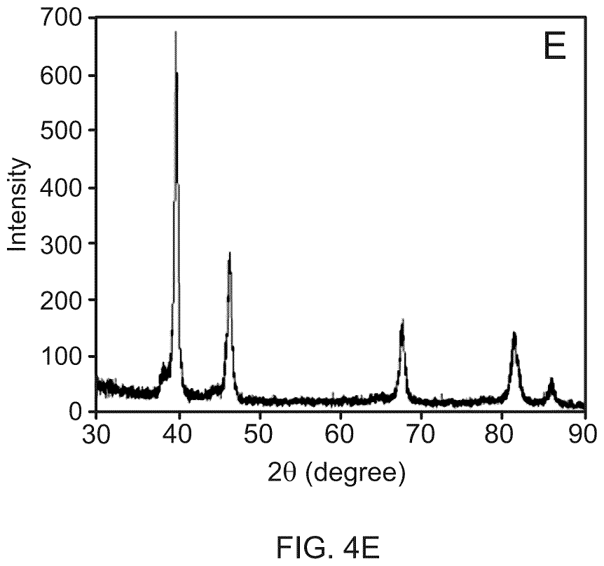

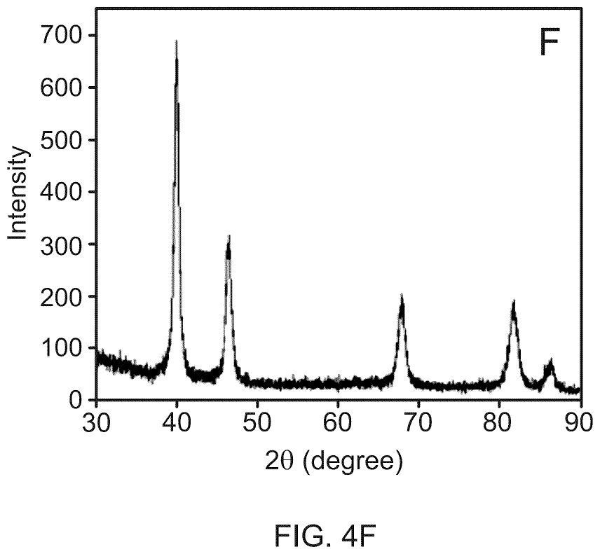

The XRD patterns of the nanoparticles prepared according to Examples II(A)-II(F) are shown in FIGS. 4A-F. In particular, FIG. 4A shows an XRD pattern of the platinum branched nanoparticles prepared according to Example II(A), FIG. 4B shows an XRD pattern of the platinum-nickel branched nanoparticles prepared according to Example II(B), FIG. 4C shows an XRD pattern of the platinum-silver nanoparticles prepared according to Example II(C), FIG. 4D shows an XRD pattern of the platinum-palladium branched nanoparticles prepared according to Example II(D), FIG. 4E shows an XRD pattern of the platinum-gold branched nanoparticles prepared according to Example II(E), and FIG. 4F shows an XRD pattern of the platinum-copper branched nanoparticles prepared according to Example II(F).

FIG. 4A confirms the presence of platinum ultrasmall nanoparticles exhibiting a face-centered cubic structure. The Pt (111) peak is situated at 39.8 degrees, and it is evident that (111) peaks of platinum-nickel, platinum-palladium, and platinum-copper extend beyond 39.8 degrees, indicating the formation of alloying nanoparticles as shown in FIGS. 4B, 4D, and 4F respectively. In FIG. 4E, it is evident that the full width at half-maximum (FWHM) of the peaks corresponding to the gold phase is larger than that of the platinum nanoparticles. This observation affirms that the size of the gold crystallites is smaller than that of the platinum nanoparticles. It is worth noting that the (111) plane of the platinum-palladium alloy closely aligns with the (111) plane of palladium (at 40.119°, JCPDF 46-1043), as shown in FIG. 4D. Additionally, platinum-gold and platinum-silver exhibit a small peak near 38 degrees (2 theta), indicating the growth of silver or gold on the surface of branched platinum.

### Example V: Sensor Fabrication

First, hydrophilic metal nanostructure inks were prepared by dispersing 10 mg of the nanoparticles prepared according to Examples II(A)-II(F) in 2.0 mL of acetic acid each. The mixtures were then stirred for 12 hours. The products were then separated by centrifuging at 4000 rpm for 2 minutes, and the supernatant was discarded. Then, 5 mL of DI water was then added to the sediment, and the mixture was again centrifuged at 4000 rpm for 5 minutes. This washing procedure was repeated twice to remove residue acid and surfactant. The resulting hydrophilic metal nanostructure inks were then applied to rectangle silicon chips and dried at room temperature such that one chip contained eight films, each film forming a resistor in a circuit with two electrodes. The two-electrode distance was 500 μm.

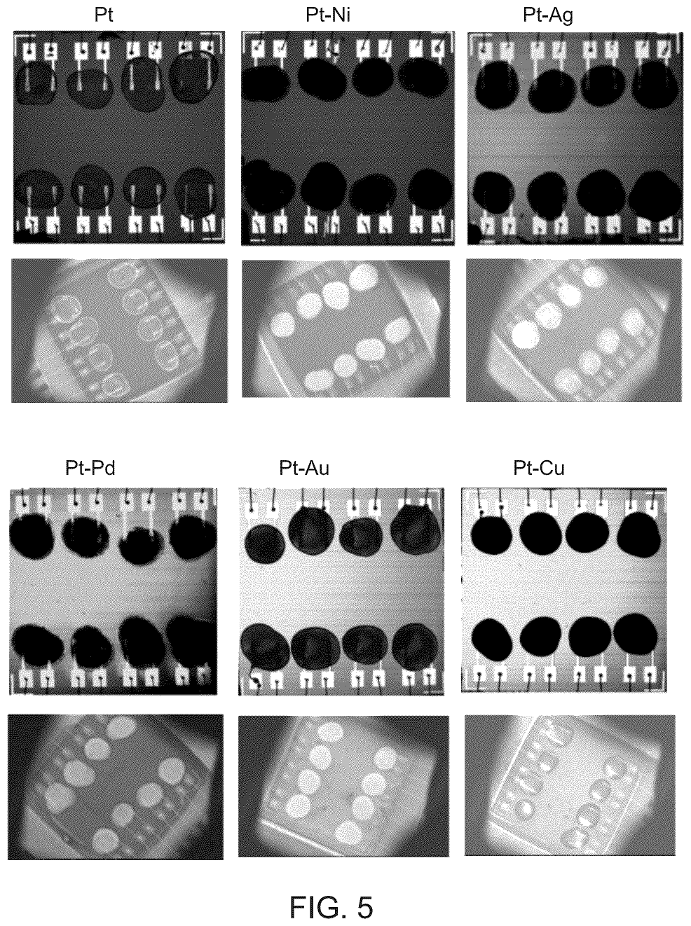

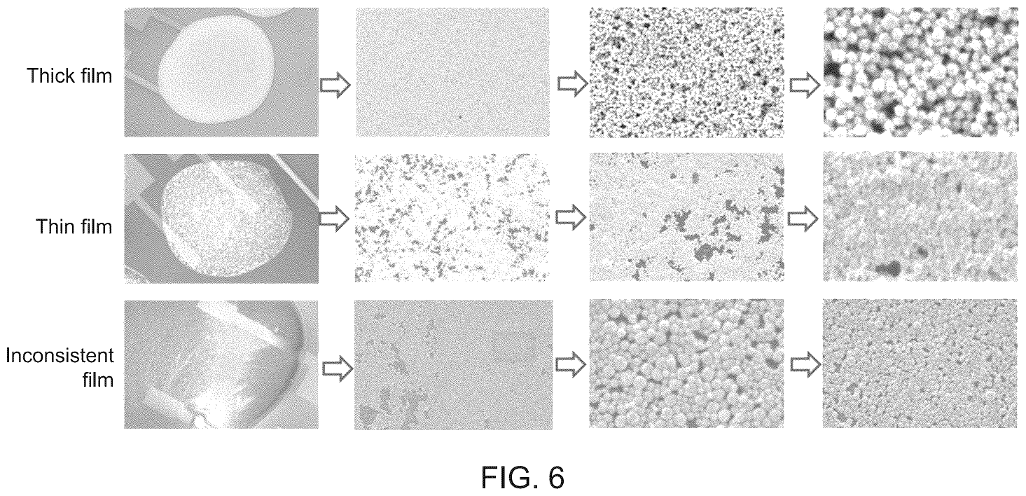

The surface morphologies of the films were investigated by a scanning electron microscope (SEM, QUANTA FEG 650) from FEI with a field emitter as electron source and with a voltage of 40 kV. FIG. 5 shows digital and SEM images of the resulting sensors. FIG. 6 shows SEM images of the sensor films with different thicknesses and consistencies. As shown in FIG. 6, the sensor films were uniform and consistent with monodispersed platinum-based bimetallic branched nanoparticles.

### Example VI: Sensor Performance

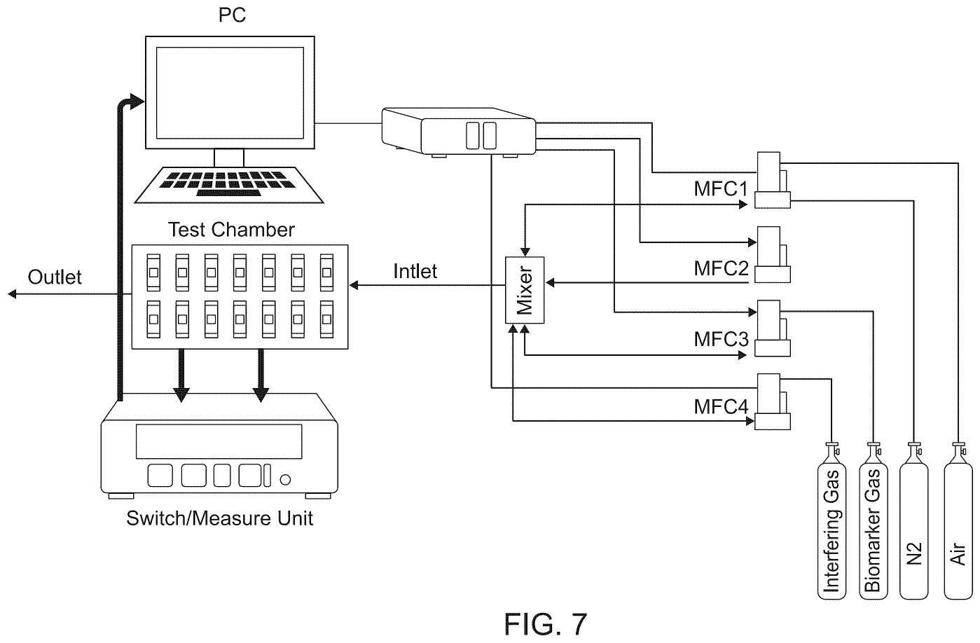

In order to assess the sensing performance of the sensors prepared according to Example V, a total of 480 sensors were tested. These sensors were arranged in an array consisting of ten chips, with each chip containing eight sensors. The measurement setup for the sensors is depicted in FIG. 7. To collect the resistance data from the sensors, the Keysight 34980A was utilized, which provided 112 channels for measurement. Additionally, the gas mixing was carefully regulated using the BROOKS SMART Interface.

The gas sensing measurement setup was put in a dry, air-conditioned environment with a temperature of about 24° C. with a consistent flow rate of the gases. Gas sensing measurements were simultaneously performed with the sensors placed inside the test chamber for each material set. The measurements were performed in two sets of conditions: in the presence of acetone, isoprene, methanol, and/or nitric oxide at various concentrations from 20 to 700 ppb, and in the presence of NH₃, ethanol, formaldehyde, NO, CO, CO₂, H₂, methane, propane, and/or acetone at various concentrations from 1 to 100 ppm. The gas concentration was set by diluting a calibration gas stabilized in nitrogen, employing two mass flow controllers (Brooks, GFC17 type).

The response-recovery time of all the sensors was measured during exposure to the gases under investigation at room temperature. To achieve the desired target levels, the different gas concentrations were prepared by diluting the gases with nitrogen using the continuous volumetric flow mixing approach. The dynamic concentration range of 20 ppb to 1000 ppm was attained through pre-dilution. The following formula was employed, where Cₛₑₜ represents the concentration set-point, C_{cylinder} denotes the concentration of the test gas in the bottle, V_{cylinder} corresponds to the flow rate from the gas bottle controlled by the mass flow controller (MFC), V_{dilution} represents the carrier flow used for pre-dilution, Vₜₑₛₜ indicates the flow rate of the pre-diluted test gas, and Vₜₒₜₐₗ represents the total flow rate through the sensor chamber.

$$\frac{{C}_{set}}{{C}_{cylinder}} = \frac{{V}_{cylinder}}{{V}_{cylinder} + {V}_{dilution}} \times \frac{{V}_{test}}{{V}_{total}}$$

Throughout all the gas sensing experiments, a constant total flow rate of 200 standard cubic centimeters per minute (sccm) into the sensor chamber was maintained. Additionally, repeatability and long-term stability are crucial factors that hold significance for practical applications. To assess these parameters, the response of all sensors for ten consecutive days were continuously recorded, while keeping the operating conditions unchanged. Furthermore, evaluations of the fabricated sensors after two months were conducted.

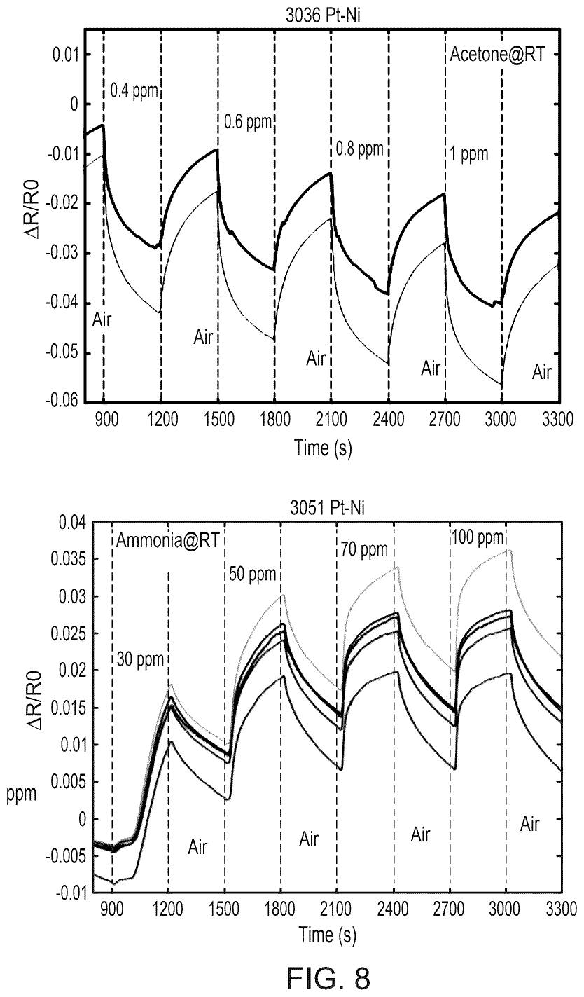

FIG. 8 shows the response of the platinum-nickel nanoparticle-based gas sensors to acetone and ammonia. As shown, when exposed to the acetone or ammonia gas, the resistance of the sensor film increases. When the gas flow stops, the resistance decreases to its starting value, indicating good, rapid recovery.

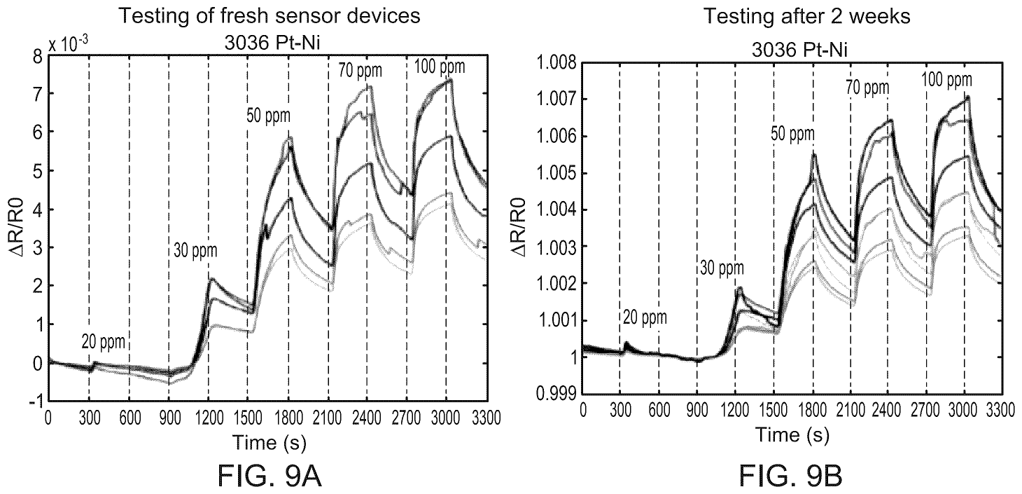

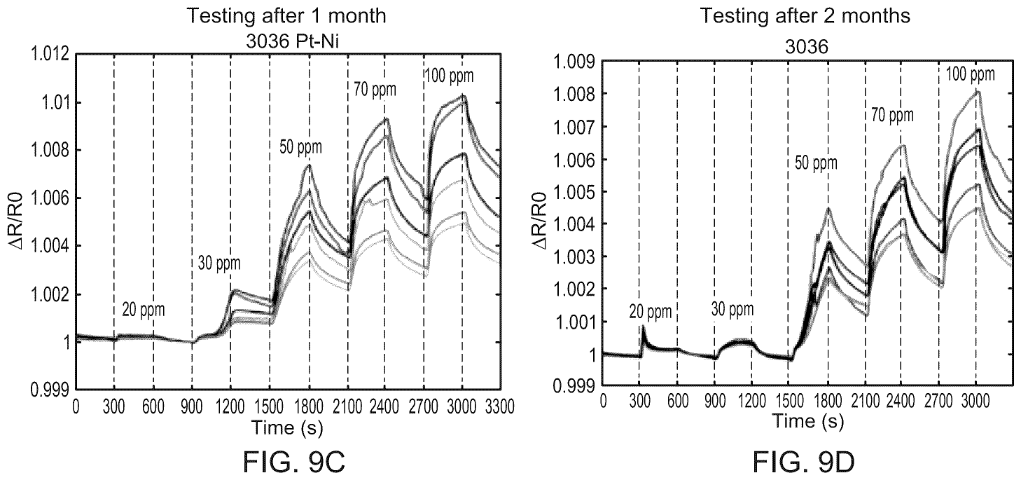

FIGS. 9A-9B show the results of the stability investigation of the platinum-nickel nanoparticle-based gas sensors for detecting ammonia. The average long-term drift stability can be estimated using Equation 1 (below), wherein differences between the initial and final days of the series (at least two months from the start of the experiment with N=8) reflect the same trend observed in sensor responses, as depicted in FIGS. 9A-9B. The contribution of long-term stability to the uncertainty of sensor measurements is estimated using Equation 2 (below), if a rectangular distribution is assumed. Equation 3 (below) is applied if a normal distribution is observed.

$${S}_{r} = \sqrt{\frac{\sum {( {R}_{i} - {R}^{-} )}^{2}}{N - 1}} \qquad Eq . ( 1 )$$

$${D}_{ls} = \frac{{\sum }_{1}^{N - 3} \vert {R}_{s , after} - {R}_{s , before} \vert}{N - 3} \qquad Eq . ( 2 )$$

$$u ( {D}_{ls}^{2} ) = {S}^{2} = \frac{{D}_{ls}^{2}}{3} \qquad Eq . ( 3 )$$

For platinum-nickel nanoparticle-based films, the standard deviation of sensor response at 100 ppm ammonia was determined to be 0.02. Consequently, the long-term drift stability was estimated at 0.03, leading to an estimated uncertainty of sensor measurement of 3%.

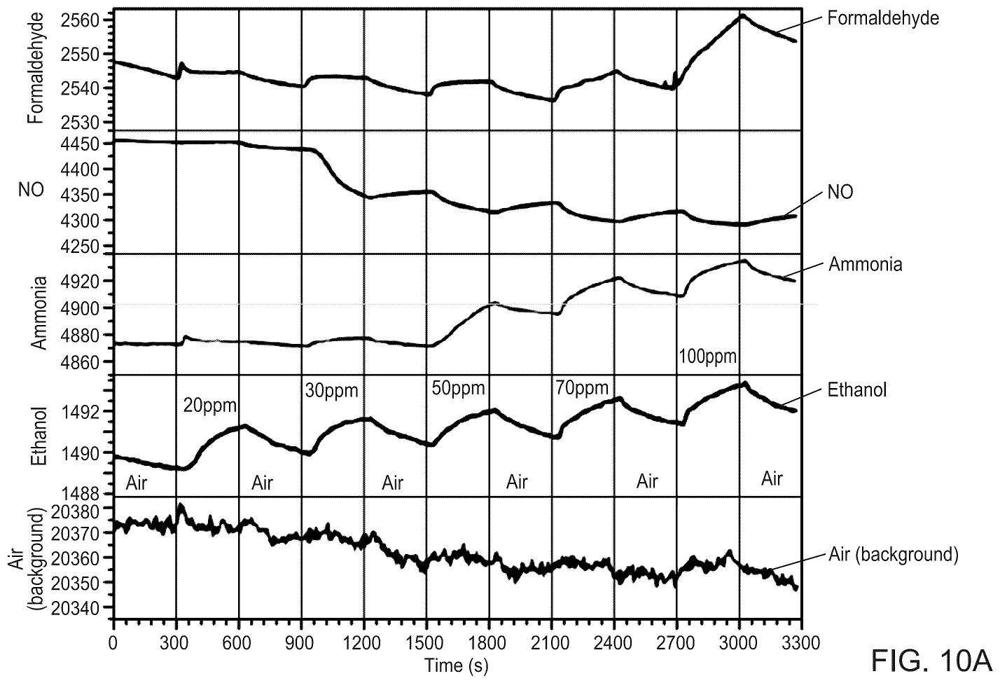

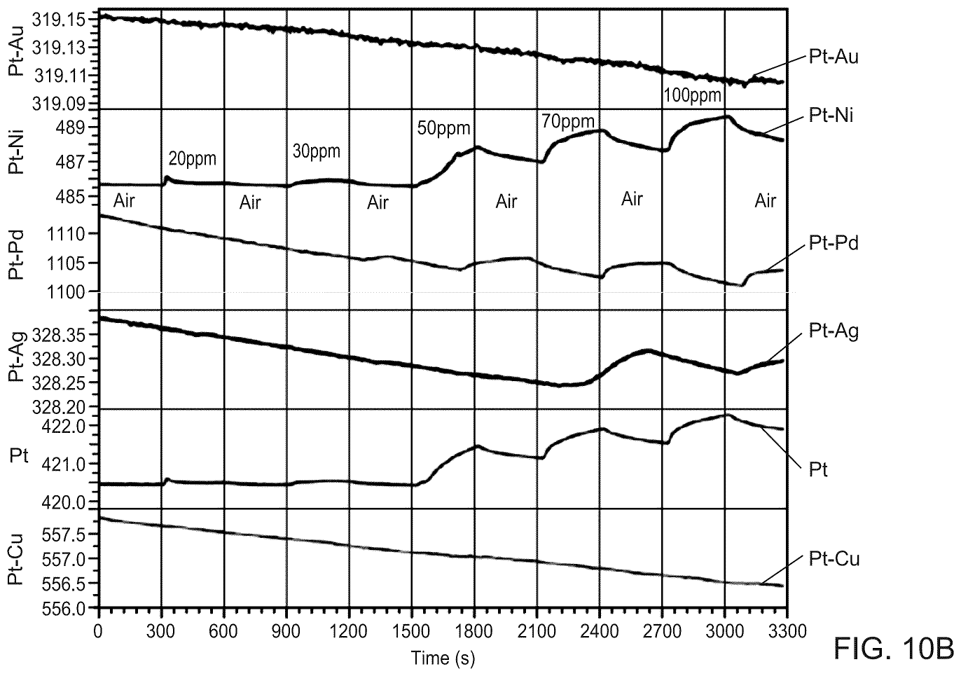

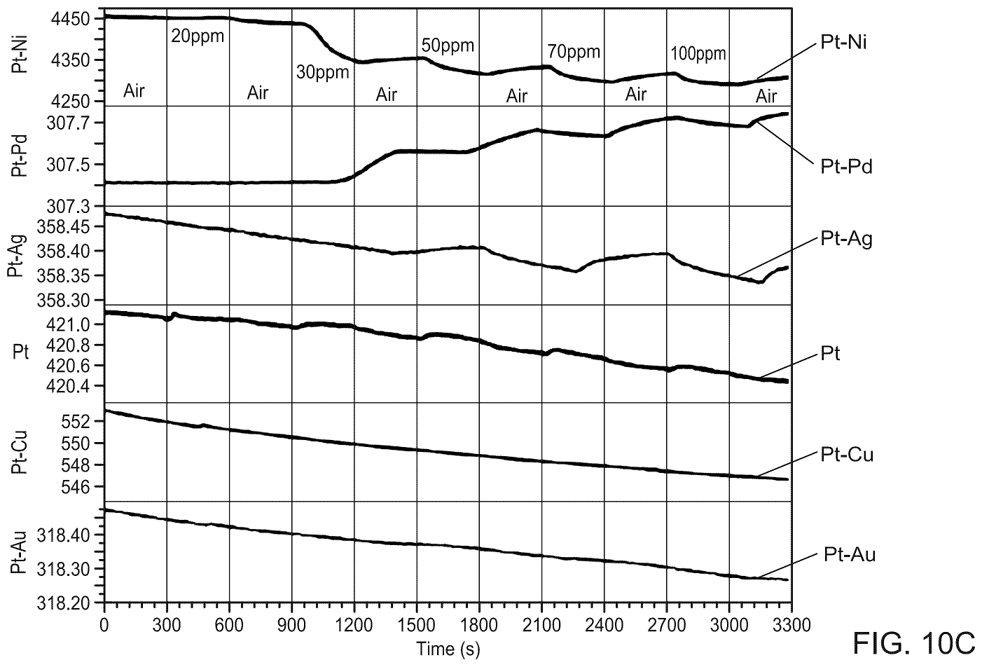

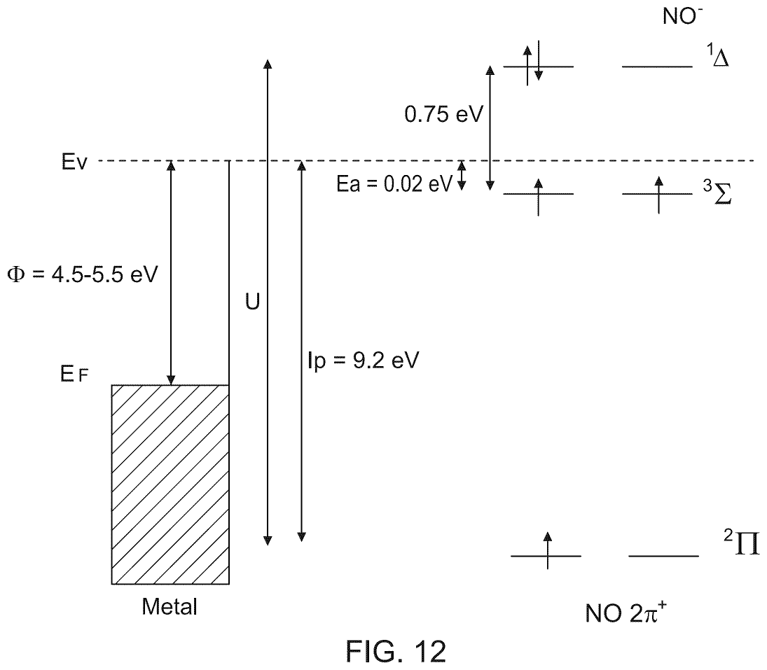

The overall response (ΔR/R₀) was measured and normalized to the background response under air. FIG. 10 shows the typical dynamic response of the sensors to the tested VOCs. Specifically, FIG. 10A compares the resistance changes of the platinum-nickel nanoparticle-based sensor upon exposure to formaldehyde, ammonia, nitric oxide, and ethanol, with air as the background gas. The resistance of the platinum-nickel nanoparticle-based film increases towards formaldehyde, ammonia, and ethanol with a linear relationship to the gas concentration increase. However, the sensor resistance decreases with exposure to nitric oxide gas. Here, nickel nanoparticles' interactions with VOCs define the response mechanisms. (See all the discussion of FIG. 12.) FIG. 10B and FIG. 10C show that ammonia and nitric oxide are sensitive with the sensor film composed of different metal nanoparticles, respectively.

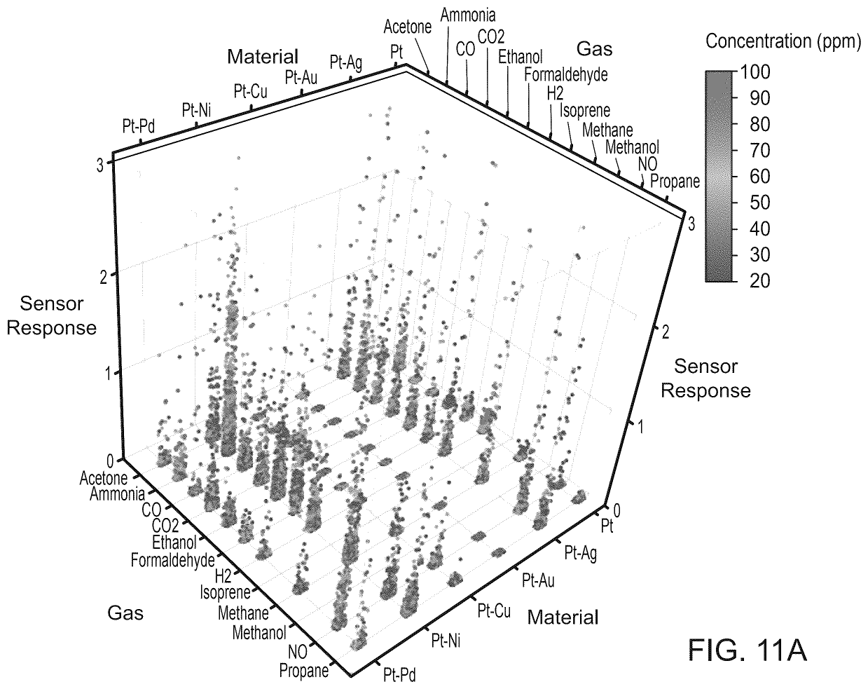

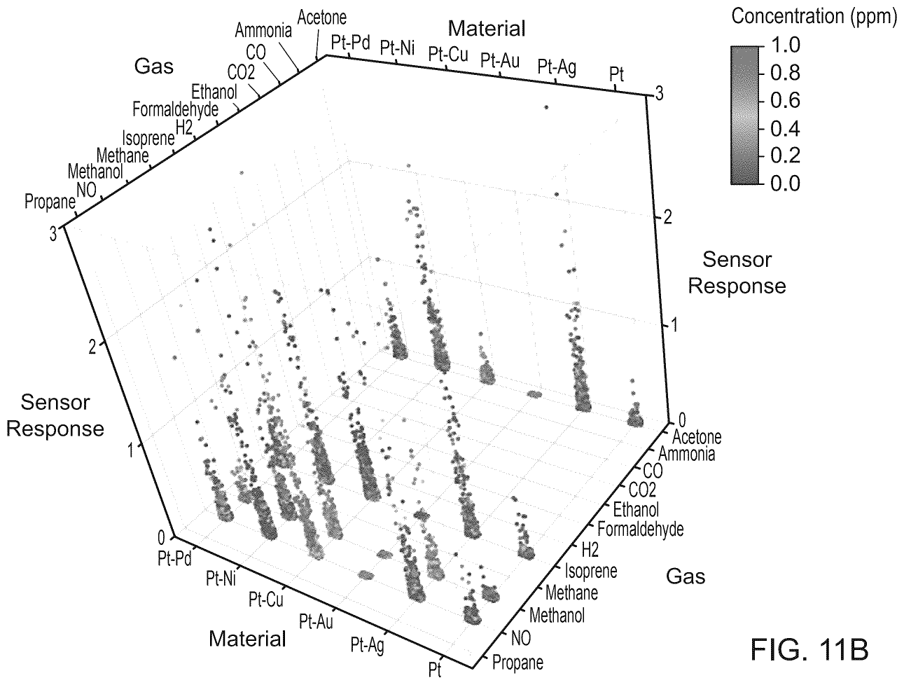

In FIGS. 11A-B, the combined response of 480 sensor nodes, containing six different sensing materials, was examined when exposed to 12 VOCs that serve as biomarkers in human breath. Among the testing gases in the ppm range, platinum-nickel and platinum-silver nanoparticles demonstrated the most significant response. Particularly, platinum-nickel nanoparticles exhibited exceptional sensitivity to formaldehyde at low concentrations (indicated by the blue dots), achieving the highest sensitivity value of 2.1 at 100 ppm ammonia. (See FIG. 11A.) On the other hand, platinum-palladium nanoparticles displayed a notable preference for responding to nitric oxide (NO), with a sensitivity as high as 2.9 at 50 ppm. It is important to highlight that the sensor array exhibited a concentration-dependent response to the VOCs. This implies that for different levels of the same VOC, a specific sensing material may show heightened sensitivity to that particular gas. By systematically adjusting the concentration from 1 ppb to 100 ppm, the collective sensor nodes array can effectively detect a range of VOCs present in breath.

FIG. 11B shows the collective sensor response to isoprene, NO, methanol, and acetone at the clinical level (ppb range). Notably, platinum-nickel nanoparticles exhibited the highest response to all four VOCs without differentiation. In contrast, platinum-copper nanoparticles displayed a notable preference, particularly responding well to 200 ppb isoprene and 1 ppm NO, with response values of 1.1 and 1.5, respectively. Out of all the materials, platinum-copper nanoparticles displayed the highest sensitivity, recording at 1.7 for 200 ppb methanol, which is known to be one of the most challenging VOCs to detect. Intriguingly, platinum-silver nanoparticles demonstrated their highest response at the lowest concentration of acetone, registering at 2.8 for 20 ppb. Despite numerous reports suggesting that gold nanoparticles can serve as selective gas sensors, this study did not yield any discernible response to the 12 tested VOCs. It was determined that specific sensors are generally capable of differentiating VOCs' type and concentration. This indicates the potential of individual sensors to respond to specific VOCs, suggesting that an array of sensors could be sensitive to a wide range of biomarkers.

The valence-energy diagram shown in FIG. 12 illustrates the energy levels of gas-phase NO in comparison to a metal surface. The diagram depicts the energy levels of gas-phase NO in the valence region alongside a metal surface with a work function of ϕ ranging from 4.5 to 5.5 eV for the metal nanoparticles. Here, Eₐ and Iₚ denote the electron affinity and ionization potential of NO, respectively. The lower and higher energy levels correspond to the 2π* electrons for NO (²Π) and NO⁻(³Σ⁻), respectively. The 2π* orbital is degenerate, and the affinity pertains to the occupation of the other orbital by an additional electron in the triplet form ³Σ⁻. Alternatively, this same orbital can be occupied by an additional electron in a singlet form ¹Δ, which is energetically higher by 0.75 eV compared to the triplet state. Considering the Coulomb interaction U, representing the repulsion with an additional electron in the same orbital, it is estimated to be Iₚ−Eₐ+0.75 eV˜10 eV for an isolated NO molecule. As the NO molecule approaches the metal surface, the 2π* levels come toward the E_{F} due to the variation of the effective potential including the image-charge effect, and at the same time, the corresponding Coulomb repulsion U is reduced, leading to the loss of the magnetic polarization in most cases as energy diagram as depicted in FIG. 12. On nickel and palladium surfaces, for example, experimental studies with photoemission spectroscopy (PES) and inverse photoemission spectroscopy (IPES) showed that the 2π* states were split and located both above and below E_{F}. The sensing experiments revealed that when NO is adsorbed onto the platinum-nickel nanoparticle surface, the sensor resistance decreases. This indicates that the bonding state of the 2π* orbital is filled above the Fermi level (E_{F}). Consequently, there is electron donation from the NO 2π* orbital to the surface.

What is claimed is:

1. A chemoresistive film comprising a plurality of metal nanostructures, the plurality of metal nanostructures having a selective affinity to at least one volatile gas species,
wherein the chemoresistive film exhibits a change in electrical resistance when the at least one volatile gas species is present in an environment of the chemoresistive film at a first concentration,
wherein the first concentration is no more than about 1000 ppm.

2. The chemoresistive film according to claim 1, wherein the plurality of metal nanostructures comprise one or more of platinum, nickel, copper, palladium, silver, and gold.

3. The chemoresistive film according to claim 1, wherein the at least one volatile gas species comprises acetone, isoprene, methanol, nitric oxide, ammonia, ethanol, formaldehyde, carbon dioxide, carbon monoxide, hydrogen, methane, propane, or a combination thereof.

4. The chemoresistive film according to claim 1, wherein the plurality of metal nanostructures comprise platinum and nickel, and wherein the at least one volatile gas species comprises formaldehyde, ammonia, or a combination thereof.

5. The chemoresistive film according to claim 1, wherein the plurality of metal nanostructures comprise platinum and palladium, and wherein the at least one volatile gas species comprises nitric oxide.

6. The chemoresistive film according to claim 1, wherein the plurality of metal nanostructures comprise platinum and copper, and wherein the at least one volatile gas species comprises isoprene, nitric oxide, methanol, or a combination thereof.

7. The chemoresistive film according to claim 1, wherein the plurality of metal nanostructures comprise platinum and silver, and wherein the at least one volatile gas species comprises acetone.

8. The chemoresistive film according to claim 1, wherein the plurality of metal nanostructures comprise branched nanoparticles.

9. The chemoresistive film according to claim 1, wherein the first concentration is no more than about 500 ppm.

10. The chemoresistive film according to claim 1, wherein the first concentration is no more than about 20 ppb.

11. A sensor comprising:
one or more chemoresistive films comprising a first chemoresistive film connected to a first lead and a second lead sufficient to complete a circuit,
wherein the sensor is configured to measure electrical resistance across the chemoresistive film,
wherein the first chemoresistive film comprises a plurality of first metal nanostructures, the plurality of first metal nanostructures having a selective affinity to at least one first volatile gas species,
wherein the first chemoresistive film exhibits a change in electrical resistance when the at least one first volatile gas species is present in an environment of the sensor at a first concentration.

12. The sensor according to claim 11, wherein the plurality of first metal nanostructures comprise one or more of platinum, nickel, copper, palladium, silver, and gold.

13. The sensor according to claim 11, wherein the at least one first volatile gas species comprises acetone, isoprene, methanol, nitric oxide, ammonia, ethanol, formaldehyde, carbon dioxide, carbon monoxide, hydrogen, methane, propane, or a combination thereof.

14. The sensor according to claim 11, wherein the plurality of first metal nanostructures comprise platinum and nickel, and wherein the at least one first volatile gas species comprises formaldehyde, ammonia, or a combination thereof.

15. The sensor according to claim 11, wherein the plurality of first metal nanostructures comprise platinum and palladium, and wherein the at least one volatile gas species comprises nitric oxide.

16. The sensor according to claim 11, wherein the plurality of first metal nanostructures comprise platinum and copper, and wherein the at least one first volatile gas species comprises isoprene, nitric oxide, methanol, or a combination thereof.

17. The sensor according to claim 11, wherein the plurality of first metal nanostructures comprise platinum and silver, and wherein the at least one first volatile gas species comprises acetone.

18. The sensor according to claim 11, wherein the plurality of first metal nanostructures comprise branched nanoparticles.

19. The sensor according to claim 11, wherein the first concentration is no more than about 1000 ppm.

20. A method of making a chemoresistive film comprising:
providing a solution comprising a plurality of metal nanostructures and a solvent;
applying the solution onto a support; and
drying the solvent.
